SEQUENCE LISTING (2 sequences)

SEQ ID NO: 1 - DNA, 22 bp, Homo sapiens
  source 1..22: mol_type = genomic DNA
tggcctggagcaacaaatctga

SEQ ID NO: 2 - DNA, 22 bp, Homo sapiens
  source 1..22: mol_type = genomic DNA
accggacctcgttgtttagact

(19) United States
(12) Patent Application Publication
(10) Pub. No.: US 2025/0041417 A1
(43) Pub. Date: Feb. 6, 2025

(54) METHODS FOR CANCER IMMUNOTHERAPY

(71) Applicant: PRECISION BIOSCIENCES, INC., Durham, NC (US)

(72) Inventors: Mark JOHNSON, Durham, NC (US); Alan F. LIST, Raleigh, NC (US); Monika VAINORIUS, Cary, NC (US)

(73) Assignee: PRECISION BIOSCIENCES, INC., Durham, NC (US)

(21) Appl. No.: 18/717,789

(22) PCT Filed: Dec. 9, 2022
(86) PCT No.: PCT/US2022/081313

§ 371 Date: Jun. 7, 2024

Related U.S. Application Data
(60) Provisional application No. 63/349,885, filed on Jun. 7, 2022.
(60) Provisional application No. 63/288,511, filed on Dec. 10, 2021.

(51) Int. Cl. A61K 39/00; A61K 31/675; A61K 31/7076; A61P 35/00; C07K 14/725; C07K 16/28

(52) U.S. Cl. CPC A61K 39/464412; A61K 31/675; A61K 31/7076; A61K 39/4611; A61K 39/4631; A61P 35/00; C07K 14/7051; C07K 16/2803

(57) ABSTRACT

The present invention encompasses methods of cancer immunotherapy, and particularly methods of allogeneic cellular immunotherapy, used in populations of subjects having cancer who previously received an autologous cell therapy, had a response, and subsequently relapsed.

### CROSS-REFERENCE TO RELATED APPLICATIONS

This application claims the benefit under 35 U.S.C. § 119(e) of the earlier filing dates of U.S. Provisional Application No. 63/288,511, filed Dec. 10, 2021; and U.S. Provisional Application No. 63/349,885, filed Jun. 7, 0222, the contents of which are incorporated by reference herein in their entirety.

### REFERENCE TO AN ELECTRONIC SEQUENCE LISTING

The contents of the electronic sequence listing (P109070068W000-SEQ-NTJ.xml; Size: 2,820 bytes; and Date of Creation: Dec. 7, 2022) are herein incorporated by reference in their entirety.

### FIELD OF THE INVENTION

The invention relates to the field of oncology and cancer immunotherapy. In particular, the invention relates to allogeneic cellular immunotherapy and lymphodepletion regimens.

### BACKGROUND OF THE INVENTION

T cell adoptive immunotherapy is a promising approach for cancer treatment. The immunotherapy treatment methods disclosed herein utilize isolated human T cells that have been genetically-modified to enhance their specificity for a specific tumor associated antigen. Genetic modification may involve the expression of a chimeric antigen receptor or an exogenous T cell receptor to graft antigen specificity onto the T cell. In contrast to exogenous T cell receptors, chimeric antigen receptors derive their specificity from the variable domains of a monoclonal antibody. Thus, T cells expressing chimeric antigen receptors (CAR T cells) induce tumor immunoreactivity in a major histocompatibility complex non-restricted manner. T cell adoptive immunotherapy has been utilized as a clinical therapy for a number of cancers, including B cell malignancies (e.g., acute lymphoblastic leukemia, B cell non-Hodgkin lymphoma, acute myeloid leukemia, and chronic lymphocytic leukemia), multiple myeloma, neuroblastoma, glioblastoma, advanced gliomas, ovarian cancer, mesothelioma, melanoma, prostate cancer, pancreatic cancer, and others.

Despite its potential usefulness as a cancer treatment, adoptive immunotherapy with CAR T cells has been limited, in part, by expression of the endogenous T cell receptor on the cell surface. CAR T cells expressing an endogenous T cell receptor may recognize major and minor histocompatibility antigens following administration to an allogeneic patient, which can lead to the development of graft-versus-host-disease (GVHD). As a result, clinical trials have largely focused on the use of autologous CAR T cells, wherein a patient's T cells are isolated, genetically-modified to incorporate a chimeric antigen receptor, and then re-infused into the same patient. An autologous approach provides immune tolerance to the administered CAR T cells; however, this approach is constrained by both the time and expense necessary to produce patient-specific CAR T cells after a patient's cancer has been diagnosed.

Thus, it would be advantageous to develop “off the shelf” CAR T cells, prepared using T cells from a third party, healthy donor, that have reduced expression, or have no detectable cell surface expression of an endogenous T cell receptor (e.g., an alpha/beta T cell receptor) and do not initiate GvHD upon administration. Such products could be generated and validated in advance of diagnosis and could be made available to patients as soon as necessary. Therefore, a need exists for the development of allogeneic CAR T cells, and CAR T cell administration regimens (i.e., including lymphodepletion regimens) that are effective for treatment.

T cell function and number are impaired by prior therapy precluding the generation of an autologous (auto) CAR T product in up to 11% of candidates (Jacobson C A, et al. J Clin Oncol 2020; 38:3095, Nastoupil L J, et al. J Clin Oncol 2020; 38:3119). Chemotherapy adversely impacts surviving T cells and reduces the frequency of early-lineage T cells necessary for CAR T cell expansion as a result of mitochondrial damage & metabolic deficiencies that skew phenotype and impair proliferation and cytotoxic capacity of adoptive cell therapies (Das R K, et al. Blood Adv 2020; 4: 4653; Deng Q, et al. Nat Med. 2020 December; 26(12):1878-1887; Lee D W, et al. Lancet. 2015; 385:517-28; Singh N, et al. Sci Transl Med. 2016; 8:320.r). Among those who receive an autologous CAR-T infusion, 30 to 48% of patients do not respond while approximately 60% of responders experience relapse of disease (Schuster S J, et al. N Engl J Med 2019; 380:45-56; SermerD. Et al. Blood Adv 2020; 4: 4669). CD19-positive relapse relates in part to poor functional attributes of CAR T effectors leading to reduced expansion, low potency and limited CAR T persistence (Maude S L, et al. N Engl J Med 2014; 371:1507-17; Gardner R, et al. Blood 2018; 132:4022; Locke F L, et al. Blood Adv 2020; 4: 4898; Ayuk F A, et al. Blood Adv 2021; 5:2523; Biasco L, et al. Nat Cancer 2021; 2:629-642; Finney O C, et al. J Clin Invest. 2019; 129(5):2123-2132; Fraietta J A, et al. Nat Med. 2018 May; 24(5): 563-571; Rossi J, et al. Blood. 2018; 132(8):804-814). There are currently no FDA approved therapeutics for patients who progress following auto-CAR T therapy, a population with poor prognosis and median OS of 3-6 months (Spiegel J Y, et al. Blood 2021; 137:1832; Locke F L, et al. J Clin Oncol 2020; 38, no. 15_suppl (May 20, 2020): 2012). Therefore, there is a need for effective therapies for patients who progress (i.e., relapse) following auto-CAR T therapy.

### SUMMARY OF THE INVENTION

Accordingly, in one aspect, the invention provides a method for reducing the number of cancer cells in a subject, the method comprising: (a) administering to the subject a lymphodepletion regimen that comprises one or more chemotherapeutic lymphodepletion agents; and (b) administering to the subject an effective dose of a pharmaceutical composition comprising a population of human immune cells; wherein a plurality of the human immune cells are genetically-modified human immune cells that express an engineered antigen receptor that has specificity for an antigen on the cancer cells, wherein the lymphodepletion regimen is administered prior to administration of the pharmaceutical composition, wherein the number of cancer cells is reduced in the subject, and wherein the subject was previously administered an autologous cell therapy, achieved a response to the autologous cell therapy, and subsequently relapsed, all prior to (a) and (b).

In another aspect, the invention provides a method for treating a cancer in a subject who has relapsed following an autologous cell therapy, the method comprising: (a) administering to the subject a lymphodepletion regimen that comprises one or more chemotherapeutic lymphodepletion agents; and (b) administering to the subject an effective dose of a pharmaceutical composition comprising a population of human immune cells; wherein a plurality of the human immune cells are genetically-modified human immune cells that express an engineered antigen receptor that has specificity for an antigen on cancer cells, wherein the lymphodepletion regimen is administered prior to administration of the pharmaceutical composition, and wherein the subject was previously administered an autologous cell therapy, achieved a response to the autologous cell therapy, and subsequently relapsed, all prior to (a) and (b).

In some embodiments, the human immune cells are not derived from the subject (i.e., such human immune cells are allogeneic).

In some embodiments, the method is a method of immunotherapy for treating cancer in the subject.

In some embodiments, the autologous cell therapy was directed against the antigen on the cancer cells (i.e., the same antigen being targeted by the present method). In some embodiments, the autologous cell therapy was directed against a different antigen than the antigen on the cancer cells by the present method.

In some embodiments, the subject achieved a partial response to the autologous cell therapy. In some embodiments, the subject achieved a complete response to the autologous cell therapy. In some embodiments, the subject received a stem cell transplant prior to relapse. In some embodiments, the subject received an allogeneic stem cell transplant prior to relapse.

In some embodiments, the autologous cell therapy comprises an autologous chimeric antigen receptor (CAR) T cell therapy. In some embodiments, the autologous cell therapy comprises an autologous CAR natural killer (CAR NK) cell therapy. In some embodiments, the autologous cell therapy comprises an autologous T cell therapy comprising administration of T cells expressing an exogenous TCR. In some embodiments, the autologous cell therapy comprises an autologous NK cell therapy comprising administration of NK cells expressing an exogenous TCR.

In some embodiments, the method is performed within about 15 days after relapse following the autologous cell therapy. In some embodiments, the method is performed within about 30 days after relapse following the autologous cell therapy. In some embodiments, the method is performed within about 45 days after relapse following the autologous cell therapy. In some embodiments, the method is performed within about 60 days after relapse following the autologous cell therapy. In some embodiments, the method is performed within about 75 days after relapse following the autologous cell therapy. In some embodiments, the method is performed within about 90 days after relapse following the autologous cell therapy. In some embodiments, the method is performed within about 120 days after relapse following the autologous cell therapy. In some embodiments, the method is performed within about 150 days after relapse following the autologous cell therapy. In some embodiments, the method is performed within about 180 days after relapse following the autologous cell therapy. In some embodiments, the method is performed within about one year after relapse following the autologous cell therapy.

In some embodiments, the subject achieves a response after the method (i.e., a clinical response). In some embodiments, the response is a partial response. In some embodiments, the response is a complete response. In some embodiments, the response persists for at least 28 days. In some embodiments, the response persists for about 45 days. In some embodiments, the response persists for at least 60 days. In some embodiments, the response persists for about 75 days. In some embodiments, the response persists for at least 90 days. In some embodiments, the response persists for about 120 days. In some embodiments, the response persists for at least 150 days. In some embodiments, the response persists for up to one year or longer.

In some embodiments, the response to the method persists longer than the response to the autologous cell therapy.

In some embodiments, the genetically-modified human immune cells expand more rapidly in the subject compared to a control subject that has not previously received the autologous cell therapy. In some embodiments, the genetically-modified human immune cells expand to achieve a higher area under the curve (AUC) in the subject compared to a control subject that has not previously received the autologous cell therapy. In some embodiments, the lymphodepletion regimen reduces the number of endogenous T cells to a lower number in the subject compared to a control subject that has not previously received autologous cell therapy. In some embodiments, the lymphodepletion regimen reduces the number of endogenous T cells for a longer period of time in the subject compared to a control subject that has not previously received autologous cell therapy.

In some embodiments, the cancer is a hematological cancer. In some embodiments, the cancer is a cancer of B cell origin or multiple myeloma. In some embodiments, cancer of B cell origin is acute lymphoblastic leukemia (ALL), chronic lymphocytic leukemia (CLL), small lymphocytic lymphoma (SLL), or non-Hodgkin lymphoma (NHL). In some embodiments, the cancer is mantle cell lymphoma (MCL) or diffuse large B cell lymphoma (DLBCL). In some embodiments, the cancer is a solid tumor cancer.

In some embodiments, the engineered antigen receptor is a CAR or an exogenous TCR having specificity for the antigen. In some embodiments, the antigen is CD19.

In some embodiments, the human immune cells are human T cells. In some embodiments, the human immune cells are human NK cells.

In some embodiments, the genetically-modified human immune cells comprise an inactivated TCR alpha gene. In some embodiments, the genetically-modified human immune cells comprise an inactivated TCR alpha constant region (TRAC) gene. In some embodiments, the genetically-modified human immune cells comprise an inactivated TCR beta gene. In some embodiments, the genetically-modified human immune cells comprise an inactivated TCR beta constant region (TRBC) gene.

In some embodiments, a transgene encoding the engineered antigen receptor is inserted into the genome of the genetically-modified immune cells. In some embodiments, the transgene is inserted within a TCR alpha gene. In some embodiments, the transgene is inserted within a TRAC gene. In some embodiments, the transgene is inserted within a TCR beta gene. In some embodiments, the transgene is inserted within a TRBC gene. In some such embodiments where the transgene is inserted within a gene, the transgene disrupts expression of the gene (e.g., the TCR alpha gene, the TRAC gene, the TCR beta gene, or the TRBC gene).

In some embodiments, the transgene is positioned in the TRAC gene within SEQ ID NO: 1. In some embodiments, the transgene is positioned in the TRAC gene between nucleotides 13 and 14 of SEQ ID NO: 1.

In some embodiments, the genetically-modified human immune cells do not have detectable cell surface expression of an endogenous alpha/beta TCR. In some embodiments, the genetically-modified human immune cells do not have detectable cell surface expression of endogenous CD3.

In some embodiments, the population of human immune cells comprises one or more of the following characteristics: (i) the genetically-modified human immune cells represent between about 40% and 75% of cells in the population of human immune cells; (ii) the genetically-modified human immune cells are T cells, and the ratio of CD4+ genetically-modified human T cells to CD8+ genetically-modified human T cells in the population is between about 0.3 and about 4.2; (iii) the genetically-modified human immune cells are T cells, and the percentage of CD4+ genetically-modified human T cells in the population that are also CCR7+ is between about 23% to about 60%; and (iv) the genetically-modified human immune cells are T cells, and the percentage of CD8+ genetically-modified human T cells in the population that are also CCR7+ is between about 14% and about 60%. In some such embodiments, the population of human immune cells can comprise any combination of the recited characteristics including: (i) alone; (ii) alone; (iii) alone; (iv) alone; a combination of (i) and (ii); a combination of (i) and (iii); a combination of (i) and (iv); a combination of (ii) and (iii); a combination of (ii) and (iv); a combination of (iii) and (iv); a combination of (i), (ii), and (iii); a combination of (i), (ii), and (iv); a combination of (i), (iii), and (iv); or a combination of (ii), (iii), and (iv).

In some embodiments, the genetically-modified human immune cells represent between about 50% and about 70% of the human immune cells in the population. In some embodiments, the genetically-modified human immune cells represent between about 55% and about 70% of the human immune cells in the population. In some embodiments, the genetically-modified human immune cells represent between about 58% and about 69% of the human immune cells in the population.

In some embodiments, no more than about 0.5% of the human immune cells in the population have detectable cell surface expression of CD3. In some embodiments, no more than about 0.3% of the human immune cells in the population have detectable cell surface expression of CD3. In some embodiments, no more than about 0.2% of the human immune cells in the population have detectable cell surface expression of CD3. In some embodiments, no more than about 0.1% of the human immune cells in the population have detectable cell surface expression of CD3. In some embodiments, no human immune cells in the population have detectable cell surface expression of CD3.

In some embodiments, the ratio of CD4+ genetically-modified human T cells to CD8+ genetically-modified human T cells in the population is between about 0.3 and about 4.2. In some embodiments, the percentage of CD4+ genetically-modified human T cells in the population that are also CCR7+ is between about 20% to about 70%. In some embodiments, the percentage of CD4+ genetically-modified human T cells in the population that are also CCR7+ is between about 23% to about 60%. In some embodiments, the percentage of CD8+ genetically-modified human T cells in the population that are also CCR7+ is between about 10% and about 60%. In some embodiments, the percentage of CD8+ genetically-modified human T cells in the population that are also CCR7+ is between about 14% and about 60%.

In some embodiments, the one or more chemotherapeutic agents comprises an alkylating agent. In some embodiments, the alkylating agent is cyclophosphamide.

In some embodiments, the lymphodepletion regimen comprises administering the alkylating agent (e.g., cyclophosphamide) at a dose of about 400 to about 1500 mg/m²/day.

In some embodiments, the lymphodepletion regimen comprises administering the alkylating agent (e.g., cyclophosphamide) at a dose of about 500 to about 1000 mg/m²/day. In some embodiments, the lymphodepletion regimen comprises administering the alkylating agent (e.g., cyclophosphamide) at a dose of about 600 to about 800 mg/m²/day. In some embodiments, the lymphodepletion regimen comprises administering the alkylating agent (e.g., cyclophosphamide) at a dose of about 500 mg/m²/day. In some embodiments, the lymphodepletion regimen comprises administering the alkylating agent (e.g., cyclophosphamide) at a dose of about 750 mg/m²/day. In some embodiments, the lymphodepletion regimen comprises administering the alkylating agent (e.g., cyclophosphamide) at a dose of about 1000 mg/m²/day.

In some embodiments, the one or more chemotherapeutic agents comprises a nucleoside analog. In some embodiments, the nucleoside analog is fludarabine.

In some embodiments, the lymphodepletion regimen comprises administering the nucleoside analog (e.g., fludarabine) at a dose of about 10 to about 40 mg/m²/day. In some embodiments, the lymphodepletion regimen comprises administering the nucleoside analog (e.g., fludarabine) at a dose of about 20 to about 40 mg/m²/day. In some embodiments, the lymphodepletion regimen comprises administering the nucleoside analog (e.g., fludarabine) at a dose of about 30 to about 40 mg/m²/day. In some embodiments, the lymphodepletion regimen comprises administering the nucleoside analog (e.g., fludarabine) at a dose of about 30 mg/m²/day.

In some embodiments, the lymphodepletion regimen comprises administering the alkylating agent (e.g., cyclophosphamide) at a dose of about 500 mg/m²/day and the nucleoside analog (e.g., fludarabine) at a dose of about 30 mg/m²/day. In some embodiments, the lymphodepletion regimen comprises administering the alkylating agent (e.g., cyclophosphamide) at a dose of about 750 mg/m²/day and the nucleoside analog (e.g., fludarabine) at a dose of about 30 mg/m²/day. In some embodiments, the lymphodepletion regimen comprises administering the alkylating agent (e.g., cyclophosphamide) at a dose of about 1000 mg/m²/day and the nucleoside analog (e.g., fludarabine) at a dose of about 30 mg/m²/day.

In some embodiments, the lymphodepletion regimen is administered to the subject daily for at least one day, or for multiple days, within 7 days prior to administration of the pharmaceutical composition.

In some embodiments, the lymphodepletion regimen comprises administering the alkylating agent (e.g., cyclophosphamide) daily starting 5 days and ending 3 days prior to administration of the pharmaceutical composition.

In some embodiments, the lymphodepletion regimen comprises administering the nucleoside analog (e.g., fludarabine) daily starting 5 days and ending 3 days prior to administration of the pharmaceutical composition. In some embodiments, the lymphodepletion regimen comprises administering the nucleoside analog (e.g., fludarabine) daily starting 6 days and ending 3 days prior to administration of the pharmaceutical composition.

In some embodiments, the lymphodepletion regimen comprises administering the alkylating agent (e.g., cyclophosphamide) and the nucleoside analog (e.g., fludarabine) daily starting 5 days and ending 3 days prior to administration of the pharmaceutical composition.

In some embodiments, the lymphodepletion regimen comprises administering the alkylating agent (e.g., cyclophosphamide) daily starting 5 days and ending 3 days prior to administration of the pharmaceutical composition, and administering the nucleoside analog (e.g., fludarabine) daily starting 6 days and ending 3 days prior to administration of the pharmaceutical composition.

In some embodiments, the lymphodepletion regimen comprises administering the alkylating agent (e.g., cyclophosphamide) at a dose of about 500 mg/m²/day and the nucleoside analog (e.g., fludarabine) at a dose of about 30 mg/m²/day daily starting 5 days and ending 3 days prior to administration of the pharmaceutical composition.

In some embodiments, the lymphodepletion regimen comprises administering the alkylating agent (e.g., cyclophosphamide) at a dose of about 750 mg/m²/day and the nucleoside analog (e.g., fludarabine) at a dose of about 30 mg/m²/day daily starting 5 days and ending 3 days prior to administration of the pharmaceutical composition.

In some embodiments, the lymphodepletion regimen comprises administering the alkylating agent (e.g., cyclophosphamide) at a dose of about 1000 mg/m²/day and the nucleoside analog (e.g., fludarabine) at a dose of about 30 mg/m²/day daily starting 5 days and ending 3 days prior to administration of the pharmaceutical composition.

In some embodiments, the lymphodepletion regimen comprises administering the alkylating agent (e.g., cyclophosphamide) at a dose of about 500 mg/m²/day daily starting 5 days and ending 3 days prior to administration of the pharmaceutical composition, and administering the nucleoside analog (e.g., fludarabine) at a dose of about 30 mg/m²/day daily starting 6 days and ending 3 days prior to administration of the pharmaceutical composition.

In some embodiments, the lymphodepletion regimen comprises administering the alkylating agent (e.g., cyclophosphamide) at a dose of about 750 mg/m²/day daily starting 5 days and ending 3 days prior to administration of the pharmaceutical composition, and administering the nucleoside analog (e.g., fludarabine) at a dose of about 30 mg/m²/day daily starting 6 days and ending 3 days prior to administration of the pharmaceutical composition.

In some embodiments, the lymphodepletion regimen comprises administering the alkylating agent (e.g., cyclophosphamide) at a dose of about 1000 mg/m²/day daily starting 5 days and ending 3 days prior to administration of the pharmaceutical composition, and administering the nucleoside analog (e.g., fludarabine) at a dose of about 30 mg/m²/day daily starting 6 days and ending 3 days prior to administration of the pharmaceutical composition.

In some embodiments, the pharmaceutical composition is administered at a dose of between about 1×10⁵ and about 6×10⁶ genetically-modified human immune cells/kg. In some embodiments, the pharmaceutical composition is administered at a dose of between about 3×10⁵ and about 6×10⁶ genetically-modified human immune cells/kg. In some embodiments, the pharmaceutical composition is administered at a dose of between about 3×10⁵ and about 3×10⁶ genetically-modified human immune cells/kg. In some embodiments, the pharmaceutical composition is administered at a dose of between about 1×10⁶ and about 3×10⁶ genetically-modified human immune cells/kg.

In some embodiments, the pharmaceutical composition is administered at a dose of about 1×10⁶ genetically-modified human immune cells/kg. In some embodiments, the pharmaceutical composition is administered at a dose of about 1.5×10⁶ genetically-modified human immune cells/kg. In some embodiments, the pharmaceutical composition is administered at a dose of about 2×10⁶ genetically-modified human immune cells/kg. In some embodiments, the pharmaceutical composition is administered at a dose of about 2.5×10⁶ genetically-modified human immune cells/kg. In some embodiments, the pharmaceutical composition is administered at a dose of about 3×10⁶ genetically-modified human immune cells/kg. In some embodiments, the pharmaceutical composition is administered at a dose of about 3.5×10⁶ genetically-modified human immune cells/kg. In some embodiments, the pharmaceutical composition is administered at a dose of about 4×10⁶ genetically-modified human immune cells/kg. In some embodiments, the pharmaceutical composition is administered at a dose of about 4.5×10⁶ genetically-modified human immune cells/kg. In some embodiments, the pharmaceutical composition is administered at a dose of about 5×10⁶ genetically-modified human immune cells/kg. In some embodiments, the pharmaceutical composition is administered at a dose of about 5.5×10⁶ genetically-modified human immune cells/kg. In some embodiments, the pharmaceutical composition is administered at a dose of about 6×10⁶ genetically-modified human immune cells/kg.

In some embodiments, the pharmaceutical composition is administered at a dose of about 200×10⁶ genetically-modified human immune cells. In some embodiments, the pharmaceutical composition is administered at a dose of about 300×10⁶ genetically-modified human immune cells. In some embodiments, the pharmaceutical composition is administered at a dose of about 400×10⁶ genetically-modified human immune cells. In some embodiments, the pharmaceutical composition is administered at a dose of about 500×10⁶ genetically-modified human immune cells. In some embodiments, the pharmaceutical composition is administered at a dose of about 600×10⁶ genetically-modified human immune cells. In some embodiments, the pharmaceutical composition is administered at a dose of about 700×10⁶ genetically-modified human immune cells. In some embodiments, the pharmaceutical composition is administered at a dose of about 800×10⁶ genetically-modified human immune cells. In some embodiments, the pharmaceutical composition is administered at a dose of about 900×10⁶ genetically-modified human immune cells. In some embodiments, the pharmaceutical composition is administered at a dose of about 1×10⁷ genetically-modified human immune cells.

In some embodiments, the lymphodepletion regimen includes no greater than a minimal effective dose of a biological lymphodepletion agent. In some embodiments, the lymphodepletion regimen includes administration of a biological lymphodepletion agent in an amount no greater than 1.0 mg/kg during the 7 day period preceding administration of the pharmaceutical composition. In some embodiments, the lymphodepletion regimen does not include administration of a biological lymphodepletion agent.

In some embodiments, the lymphodepletion regimen includes administration of a biological lymphodepletion agent.

In some embodiments, the biological lymphodepletion agent is a monoclonal antibody, or a fragment thereof. In some embodiments, the monoclonal antibody, or fragment thereof, has specificity for a T cell antigen. In some embodiments, the monoclonal antibody, or fragment thereof, is an anti-CD52 monoclonal antibody, or fragment thereof, or an anti-CD3 antibody, or fragment thereof. In particular embodiments, the anti-CD52 monoclonal antibody is alemtuzumab or ALLO-647. In particular embodiments, the anti-CD3 monoclonal antibody is foralumab.

In some embodiments, the method reduces the size of a cancer or tumor in the subject.

In some embodiments, the method eradicates the cancer or tumor in the subject.

### BRIEF DESCRIPTION OF THE DRAWINGS

FIG. 1 sets forth a diagram illustrating a PBCAR0191 regimen, including screening, lymphodepletion, dosing of a composition of PBCAR0191 CAR T cells, and follow-up.

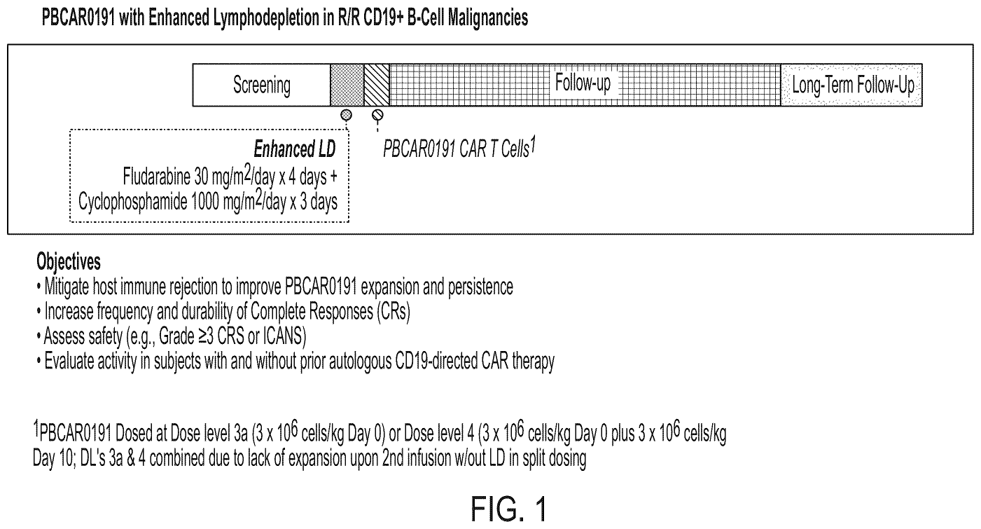

FIG. 2 sets forth a diagram illustrating the detection of PBCAR0191 CAR T cells in peripheral blood samples by molecular analysis over time. NHL and B-ALL subjects treated with enhanced lymphodepletion (eLD) followed by PBCAR0191 dosing (purple line) showed markedly improved peak CAR T cell expansion and PBCAR0191 area under the curve (AUC) compared to subjects administered standard lymphodepletion (blue line). Of note, data for five subjects included in the eLD group had previously been treated with an autologous CD19 directed CAR T cell therapy.

FIG. 3 sets forth a table describing subject demographic and cancer histology information for NHL and B-ALL subjects dosed with PBCAR0191 after receiving eLD. The enrolled subjects were comprised of a heavily pretreated population, with a median five lines of prior therapy for both disease cohorts. Importantly, five of eighteen NHL patients, and one of five B-ALL patients, had previously received an autologous CD19-directed CAR T therapy.

FIG. 4 sets forth a table describing adverse events of special interest (AESI) in subjects dosed with PBCAR0191 after receiving eLD.

FIG. 5 sets forth a table describing the overall response rate (ORR) and frequency of subjects with a complete response (CR) for all evaluable subjects, and for subjects separated by prior treatment with an autologous CD19 directed CAR T therapy. In CAR naïve subjects, an ORR and CR rate of 63% and 56% was reported. By comparison, an ORR of 100% was reported for CAR T experienced subjects, with 67% of those in CR.

FIG. 6 sets forth a table describing the overall response rate (ORR) and frequency of subjects with a complete response (CR) in NHL and B-ALL subjects dosed PBCAR0191 after receiving eLD. Of interest, ORR of greater than 70% were reported in both cohorts, with over half of subjects experiencing a CR.

FIG. 7 sets forth a diagram illustrating the duration and depth of clinical response in NHL subjects treated with PBCAR0191 after receiving eLD. Four of seventeen evaluable subjects reached assessment at Day 180 post-PBCAR0191 dosing, with a fifth subject receiving a hematopoietic cell transplantation (HCT) Day >140 post-PBCAR0191 dosing. Subjects who had previously received a stem cell transplant (purple star, n=4)), an autologous CD19 directed CAR T therapy (yellow star, n=2), or both prior stem cell transplantation and an autologous CD19 directed CAR T therapy (blue star, n=3) are designated in the diagram.

FIG. 8 sets forth a diagram illustrating the duration and depth of clinical response in B-ALL subjects treated with PBCAR0191 after receiving eLD. Three out of five subjects secured an adequate window for an allogeneic-HCT. Interestingly, one subject who had previously received an autologous CD19 direct CAR T therapy was deemed transplant ineligible but was in a minimal residual disease (MRD) negative ongoing CR for >9 months.

FIG. 9 sets forth a diagram describing initial program conclusions from subjects dosed with PBCAR0191 after receiving eLD. Specifically, heavily pre-treated subjects dosed with PBCAR0191 after receiving eLD showed markedly improved peak CAR T expansion and AUC that yielded ORR and CR rates that were comparable to autologous CAR T products. Moreover, this group included a subset that had previously relapsed after autologous CAR T cell therapy.

FIG. 10 sets forth a diagram describing initial program conclusions in relation to enrolled subjects who had previously relapsed after CD19 directed autologous CAR T cell therapy. Importantly, patients who never respond, or relapsed after response, to autologous CAR T therapy have limited therapeutic options. The initial signal seen in these subjects after PBCAR0191 dosing with eLD may provide a therapeutic option in this growing population with high unmet need.

FIG. 11 sets forth a table providing characteristics of the clinical trial materials utilized for the PBCAR0191 protocol.

FIG. 12 sets forth a diagram illustrating the duration and depth of clinical response after treatment with autologous CD19 directed CAR T therapy compared to clinical response after PBCAR0191 with eLD. Three of five evaluable subjects have a response duration after treatment with PBCAR0191 that exceeded the duration of response to autologous CAR T cell therapy. The sixth subject is 1 of 3 subjects still in on-going response after PBCAR0191 treatment.

FIG. 13 sets forth a diagram illustrating the kinetics of CD3+ host T-cells in peripheral blood samples by flow cytometric analysis. Autologous CD19 directed CAR T naïve subjects (blue line) had higher numbers of CD3+ host T cells, and showed quicker recovery of these cells, after lymphodepletion compared to subjects who had relapsed after autologous CAR T therapy (purple line).

FIG. 14 sets forth a diagram illustrating the kinetics of PBCAR0191 CAR T cells in peripheral blood samples by flow cytometric analysis. Subjects who had previously relapsed after autologous CAR T therapy (purple line) showed higher peak CAR T cell expansion and improved AUC compared to subjects who were autologous CAR T naïve (blue line).

FIG. 15 sets forth a diagram describing initial program conclusions from autologous CAR T therapy relapsed subjects subsequently dosed with PBCAR0191. In six subjects dosed with PBCAR0191 after autologous CAR T relapse, the ORR was 100% with 67% of those subjects in CR. This initial signal provides rationale to enroll additional subjects in a growing population without alternative therapeutic interventions.

FIG. 16 shows PBCAR0191 subject characteristics.

FIG. 17 shows efficacy of the lymphodepletion regimen and CAR T cell dosing in the PBCAR0191 study.

FIG. 18 shows PBCAR0191 response rates and durability.

FIG. 19 shows PBCAR0191 response rates and durability using dose level DL4b with the modified lymphodepletion regimen (mLD).

FIG. 20 shows CAR T cell expansion in vivo after dosing in the PBCAR0191 study.

FIG. 21 shows CAR T cell numbers in vivo after dosing in the PBCAR0191 study compared to CAR T cell numbers observed in patients receiving autologous CAR T cell therapy.

FIG. 22 shows the adverse events of special interest (AESI) profile in the PBCAR0191 study.

FIG. 23 shows median hematologic recovery achieved during the PBCAR0191 study.

FIG. 24 shows a summary of interim results obtained in the PBCAR0191 study.

### BRIEF DESCRIPTION OF THE SEQUENCES

SEQ ID NO: 1 sets forth the nucleic acid sequence of the TRC 1-2 recognition sequence (sense).

SEQ ID NO: 2 sets forth the nucleic acid sequence of the TRC 1-2 recognition sequence (antisense).

### DETAILED DESCRIPTION

### 1. References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the term “lymphodepletion” or “lymphodepletion regimen” refers to the administration to a subject of one or more agents (e.g., chemotherapeutic lymphodepletion agents or biological lymphodepletion agents) capable of reducing endogenous lymphocytes in the subject for immunotherapy; e.g., a reduction of one or more lymphocytes (e.g., B cells, T cells, and/or NK cells) by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control (e.g., relative to a starting amount in the subject undergoing treatment, relative to a pre-determined threshold, or relative to an untreated subject).

As used herein, the term “autologous cell therapy” refers to a therapy in which a patient's own immune cells are isolated, modified, expanded, and re-introduced into the same patient in order to achieve a therapeutic outcome, such as the treatment of a disease. The isolated immune cells can be, for example, a patient's T cells or NK cells. In particular types of autologous cell therapy, the patient's immune cells are modified to express an engineered antigen receptor, such as a CAR or an exogenous TCR which have specificity for an antigen present on the surface of the target cancer cells. Examples of such therapies include the use of a patient's T cells expressing a CAR (i.e., “autologous chimeric antigen receptor (CAR) T cell therapy”), such as YESCARTA™, KYMRIAH™, TECARTUS™, BREYANZI®, or ABECMA®. In another example, such therapies include the use of a patient's T cells expressing an exogenous TCR (i.e., “autologous T cell therapy comprising administration of T cells expressing an exogenous TCR”). In further examples, such therapies include the use of a patient's NK cells expressing a CAR (i.e., “autologous CAR natural killer (CAR NK) cell therapy”) or expressing an exogenous TCR (i.e., “autologous NK cell therapy comprising administration of NK cells expressing an exogenous TCR”).

As used herein, the term “previously administered an autologous cell therapy” means that an autologous cell therapy, including the administration of any lymphodepletion regimen and the administration of genetically-modified immune cells, was administered to a subject prior to initiation of the methods described herein. For clarity, administration of an autologous cell therapy, achieving a response to such autologous cell therapy, and relapse, all occur prior to initiation of the methods described herein.

As used herein, the term “biological lymphodepletion agent” refers to a biological material, such an antibody, antibody fragment, antibody conjugate, or the like, that can be administered as part of a lymphodepletion regimen to reduce endogenous lymphocytes in the subject for immunotherapy. In some cases, such biological lymphodepletion agents can have specificity for antigens present on lymphocytes; e.g., CD52 or CD3.

As used herein, the term “chemotherapeutic lymphodepletion agents” refers to non-biological materials, such as small molecules, that can be administered as part of a lymphodepletion regimen to reduce endogenous lymphocytes in the subject for immunotherapy. In some examples, the chemotherapeutic lymphodepleting agent can be lymphodepleting but non-myeloablative.

As used herein, the term “alkylating agent” refers to a certain class of compounds useful in chemotherapy and lymphodepletion which are antineoplastic and act by inhibiting the transcription of DNA into RNA and thereby stop protein synthesis. Alkylating agents useful in the invention can include, for example, cyclophosphamide.

As used herein, the term “nucleoside analog” refers to a certain class of compounds useful in chemotherapy and lymphodepletion, particularly those that are metabolized by deoxycytidine kinase such that they are converted from a prodrug form to an active form. Nucleoside analogs useful in the invention can include, for example, fludarabine, cytarabine, gemcitabine, and decitabine.

The terms “effective dose”, “effective amount”, “therapeutically effective dose”, or “therapeutically effective amount,” as used herein, refers to an amount sufficient to effect beneficial or desirable biological and/or clinical results. In some embodiments, the effective dose is equivalent to the suggested, recommended or allowed dose (for adults or children) provided in the drug product labeling for a biological lymphodepletion agent.

The term “minimal effective dose,” as used herein, refers to weekly administration of an amount of a pharmaceutical agent that is equivalent to 10% of the maximum weekly dose as set forth by the United States Food and Drug Administration (FDA) or the European Medicines Agency (EMA), for instance, in the labeling for any drug product(s) that comprise the agent. Thus, for a biological lymphodepletion agent, a “minimal effective dose” can be 10% of the maximum dose of the agent approved for use in lymphodepletion by the FDA or the EMA.

As used herein, the terms “treatment”, “treating”, or “treating a subject” refers to the administration of a pharmaceutical composition disclosed herein, comprising a population of human immune cells, to a subject having a disease, disorder or condition. For example, the subject can have a disease such as cancer, and treatment can represent immunotherapy for the treatment of the disease. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, a partial or complete reduction in the number of cancer cells present in the subject, and remission or improved prognosis. In some aspects, treatment includes the administration of a lymphodepletion regimen to reduce endogenous lymphocytes in the subject for immunotherapy.

As used herein, the term “immune cell” or “human immune cell” refers to any cell that is part of the immune system (innate and/or adaptive) and is of hematopoietic origin.

Non-limiting examples of immune cells include lymphocytes, B cells, T cells, monocytes, macrophages, dendritic cells, granulocytes, megakaryocytes, monocytes, macrophages, natural killer cells, myeloid-derived suppressor cells, innate lymphoid cells, platelets, red blood cells, thymocytes, leukocytes, neutrophils, mast cells, eosinophils, basophils, and granulocytes.

As used herein, the terms “human T cell” and “human T lymphocyte” are used interchangeably herein and refer to a white blood cell of the lymphocyte subtype that expresses T cell receptors on the cell membrane. T cells develop in the thymus gland and include both CD8+ T cells and CD4+ T cells, as well as natural killer T cells, memory T cells, gamma delta T cells, and any other lymphocytic cell that expresses a T cell receptor. In some cases, the human donor is not the subject treated according to the method (i.e., the T cells are allogeneic), but instead a healthy human donor. In some cases, the human donor is the subject treated according to the method. T cells, and cells derived therefrom, can include, for example, isolated T cells that have not been passaged in culture, or T cells that have been passaged and maintained under cell culture conditions without immortalization. In some cases, the human T cell is a differentiated induced pluripotent stem cell (iPSC); e.g., an iPSC derived from a human somatic cell.

As used herein, the terms “human natural killer cell” or “human NK cell” or “natural killer cell” or “NK cell” refers to a type of cytotoxic lymphocyte critical to the innate immune system. The role NK cells play is analogous to that of cytotoxic T cells in the vertebrate adaptive immune response. NK cells provide rapid responses to virally infected cells and respond to tumor formation, acting at around 3 days after infection. Human NK cells, and cells derived therefrom, include isolated NK cells that have not been passaged in culture, NK cells that have been passaged and maintained under cell culture conditions without immortalization, and NK cells that have been immortalized and can be maintained under cell culture conditions indefinitely. In some cases, the human NK cell is a differentiated induced pluripotent stem cell (iPSC); e.g., an iPSC derived from a human somatic cell.

As used herein, the term “antibody” refers to a protein, or polypeptide sequence derived from an immunoglobulin molecule which specifically binds with an antigen.

Antibodies can be polyclonal or monoclonal, multiple or single chain, or intact immunoglobulins, and may be derived from natural sources or from recombinant sources.

Antibodies can be tetramers of immunoglobulin molecules.

As used herein, the term “engineered antigen receptor” refers to an engineered receptor that confers or grafts specificity for an antigen onto an effector cell, such as a human immune cell. Engineered antigen receptors include, for example, chimeric antigen receptors (CARs) and exogenous T cell receptors, such as those described herein.

As used herein, the term “chimeric antigen receptor” or “CAR” refers to an engineered receptor that confers or grafts specificity for an antigen onto an immune effector cell (e.g., a human T cell). A chimeric antigen receptor comprises at least an extracellular ligand-binding domain or moiety, a transmembrane domain, and an intracellular domain, wherein the intracellular domain comprises one or more signaling domains and/or co-stimulatory domains.

In some embodiments, the extracellular ligand-binding domain or moiety is an antibody, or antibody fragment. In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHi domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

The intracellular stimulatory domain includes one or more cytoplasmic signaling domains that transmit an activation signal to the T cell following antigen binding. Such cytoplasmic signaling domains can include, without limitation, a CD3 zeta signaling domain.

The intracellular stimulatory domain can also include one or more intracellular co-stimulatory domains that transmit a proliferative and/or cell-survival signal after ligand binding. In some cases, the co-stimulatory domain can comprise one or more TRAF-binding domains. Such intracellular co-stimulatory domains can be any of those known in the art and can include, without limitation, those co-stimulatory domains disclosed in WO 2018/067697 including, for example, Novel 6 (“N6”). Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof.

A chimeric antigen receptor further includes additional structural elements, including a transmembrane domain that is attached to the extracellular ligand-binding domain via a hinge or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (α chain), p75 (β chain) or γ chain, subunit chain of Fc receptors (e.g., Fcy receptor III) or CD proteins such as the CD8 alpha chain. In certain examples, the transmembrane domain is a CD8 alpha domain. Alternatively, the transmembrane domain can be synthetic and can comprise predominantly hydrophobic residues such as leucine and valine.

The hinge region refers to any oligo or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcγRllla receptor or IgG1. In certain examples, the hinge region can be a CD8 alpha domain.

As used herein, a “chimeric antigen receptor T cell” or “CAR T cell” refers to a human T cell modified to comprise a transgene encoding a CAR, wherein the CAR is expressed on the cell surface of the T cell. The CAR T cell may be derived from any “human T cell,” as that term is used herein. For instance, a CAR T cell may be derived from a human T cell that has been isolated from a human donor, e.g., a human donor that is not the subject treated according to the method (i.e., the CAR T cells are allogeneic), but instead a healthy human donor.

As used herein, the terms “exogenous T cell receptor” or “exogenous TCR” refer to a TCR whose sequence is introduced into the genome of an immune effector cell (e.g., a human T cell) that may or may not endogenously express the TCR. Expression of an exogenous TCR on an immune effector cell can confer specificity for a specific epitope or antigen (e.g., an epitope or antigen preferentially present on the surface of a cancer cell or other disease-causing cell or particle). Such exogenous T cell receptors can comprise alpha and beta chains or, alternatively, may comprise gamma and delta chains. Exogenous TCRs useful in the invention may have specificity to any antigen or epitope of interest. In some examples, exogenous TCRs can include an extracellular ligand-binding domain comprising an antibody, or antibody fragment, having specificity for a target antigen. Such an antibody fragment can be, for example, a single-chain variable fragment (scFv). An “exogenous T cell receptor” or “exogenous TCR” can also refer to a cell surface TCR complex that incorporates one or more genetically-modified and/or exogenous TCR components (e.g., a TRuC; see for example, WO2016187349, WO2018026953, WO2018067993, WO2018098365, WO2018119298, and WO2021035170). Accordingly, in embodiments wherein a nucleic acid sequence encodes an “exogenous T cell receptor” or “exogenous TCR”, this can refer to a sequence encoding one or more genetically-modified and/or exogenous TCR complex components that, when expressed, associate with endogenous TCR components to form a functional modified TCR complex on the cell surface.

As used herein, a “chimeric antigen receptor natural killer cell” or “chimeric antigen receptor NK cell” or “CAR NK cell” refers to a human NK cell modified to comprise a transgene encoding a CAR, wherein the CAR is expressed on the cell surface of the NK cell. The CAR NK cell may be derived from any “human NK cell,” as that term is used herein. For instance, a CAR NK cell may be derived from a human NK cell that has been isolated from a human donor, e.g., a human donor that is not the subject treated according to the method (i.e., the CAR NK cells are allogeneic), but instead a healthy human donor.

As used herein, the term “T cell receptor alpha gene” or “TCR alpha gene” refer to the locus in a T cell which encodes the T cell receptor alpha subunit. The T cell receptor alpha gene can refer to NCBI Gene ID number 6955, before or after rearrangement. Following rearrangement, the T cell receptor alpha gene comprises an endogenous promoter, rearranged V and J segments, the endogenous splice donor site, an intron, the endogenous splice acceptor site, and the T cell receptor alpha constant region locus, which comprises the subunit coding exons.

As used herein, the term “T cell receptor alpha constant region” or “TCR alpha constant region” or “TRAC” refers to a coding sequence of the T cell receptor alpha gene. The TCR alpha constant region includes the wild-type sequence, and functional variants thereof, identified by NCBI Gene ID NO. 28755.

As used herein, the term “T cell receptor beta gene” or “TCR beta gene” refers to the locus in a T cell which encodes the T cell receptor beta subunit. The T cell receptor beta gene can refer to NCBI Gene ID number 6957.

As used herein, the term “CD4+” or “CD4-positive” refers to T cells that express the surface protein CD4. Such T cells can, for example, be T helper cells. As used herein, the term “CD8+” or “CD8-positive” refers to T cells that express the surface protein CD8. Such T cells can, for example, be cytotoxic cells.

As used herein, the term “CCR7+” or “CCR7-positive” refers to T cells that express the chemokine receptor CCR7 on their cell surface. T cells which are both CD4+/CCR7+, or both CD8+/CCR7+, can represent T cell populations having a naïve/stem cell memory phenotype, which can be characterized as CD62L+/CD45RA+/CCR7+, and/or a central memory phenotype, which can be characterized as CD62L−/CD45RO+/CCR7+.

As used herein, the term “cancer” should be understood to encompass any neoplastic disease (whether invasive or metastatic) which is characterized by abnormal and uncontrolled cell division causing malignant growth or tumor. For example, this term includes, without limitation, hematological malignancies. Such hematological malignancies can include, for example as B cell malignancies such as, without limitation, acute lymphoblastic leukemia, B cell non-Hodgkin lymphoma, acute myeloid leukemia, chronic lymphocytic leukemia, small lymphocytic leukemia, mantle cell lymphoma, diffuse large B cell lymphoma, or multiple myeloma. Cancers can also include solid tumors. Such solid tumor can include, without limitation, neuroblastoma, glioblastoma, advanced gliomas, ovarian cancer, mesothelioma, melanoma, prostate cancer, pancreatic cancer, head and neck cancers, lung cancers, and breast cancers. In some embodiments, the methods result in a reduction in the number of cancer cells within the subject when compared to a control (e.g., relative to a starting amount in the subject prior to treatment according to the presently disclosed methods, relative to a pre-determined threshold, or relative to an untreated subject). That is, the number of cancer cells in the subject may be reduced by a percentage using the methods described herein. Such a reduction may be up to 5%, 10%, 20%, 30%, 40%, 50%, 60%, 70%, 80%, 90%, 95%, 96%, 97%, 98%, 99%, or up to 100%.

As used herein, “acute lymphoblastic leukemia” or “ALL” refers to a cancer of the lymphoid line of blood cells characterized by the development of large numbers of immature lymphocytes.

As used herein, “non-Hodgkin lymphoma” or “NHL” refers to a group of blood cancers that includes all types of lymphoma except Hodgkin's lymphomas.

As used herein, “chronic lymphocytic leukemia” or “CLL” refers to a type of non-Hodgkin lymphoma cancer characterized by the clonal proliferation and accumulation of neoplastic B lymphocytes in the blood and bone marrow.

As used herein “small lymphocytic leukemia” or “SLL” refers to a type of non-Hodgkin lymphoma cancer characterized by the clonal proliferation and accumulation of neoplastic B lymphocytes in the lymph nodes, and spleen.

As used herein, “mantle cell lymphoma” or “MCL” refers to a type of non-Hodgkin lymphoma cancer characterized by a CD5 positive antigen-naïve pre-germinal center B-cell within the mantle zone that surrounds normal germinal center follicles. MCL cells generally over-express cyclin D1 due to a chromosomal translocation in the DNA.

As used herein, “diffuse large B cell lymphoma” or “DLBCL” refers to a non-Hodgkin lymphoma affecting B cells that can develop in the lymph nodes or in extranodal sites (areas outside the lymph nodes) such as the gastrointestinal tract, testes, thyroid, skin, breast, bone, brain, or essentially any organ of the body.

As used herein, the terms “response,” “complete response,” “complete response with incomplete blood count recovery,” “refractory disease,” “partial response,” “disease progression” or “progressive disease,” “refractory disease,” “relapse” or “relapsed disease” each refer to assessments of disease state and response in subjects following treatment according to the methods disclosed herein. For example, the response criteria for the assessment of subjects with B-ALL are based on the NCCN Guidelines (NCCN, 2017). As described therein, a complete response is defined as no circulating blasts or extramedullary disease, no lymphadenopathy, splenomegaly, skin/gum infiltration/testicular mass/CNS involvement, trilineage hematopoiesis and <5% blasts, absolute neutrophil count (ANC) >1000/mm³, platelets >100,000/mm³, and no recurrence for 4 weeks. Complete response with incomplete blood count recovery (CRi) is defined as meeting all criteria for complete response except platelet count and/or ANC. An overall response rate (ORR) can be calculated as CR+CRi. Refractory disease can be defined as failure to achieve a complete response at the end of induction. Progressive disease can be defined as an increase of at least 25% in the absolute number of circulating or bone marrow blasts or development of extramedullary disease. Relapsed disease can be defined as the reappearance of blasts in the blood or bone marrow (>5%) or in any extramedullary site after a complete response.

For NHL, response criteria for local and central assessments of subjects with NHL are based on the revised Lugano Classification (Cheson et al, 2016), which incorporates PET-CT. A complete response (i.e., a complete metabolic response) is characterized by lymph nodes and extralymphatic sites having a score of 1, 2, or 3 with or without a residual mass on 5-point scale (5PS), and it is recognized that in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake. A complete response is further characterized by no new lesions and no evidence of fluorodeoxyglucose (FDG)-avid disease in marrow. A partial response (i.e., partial metabolic response) is characterized by lymph nodes and extralymphatic sites having a score of 4 or 5 with reduced uptake compared with Baseline and residual mass(es) of any size. At interim, these findings suggest responding disease. At end of treatment, these findings indicate residual disease. A partial response is further characterized by no new lesions and bone marrow wherein residual uptake is higher than uptake in normal marrow but reduced compared with baseline. No response or stable disease (i.e., no metabolic response) is characterized by target nodes/nodal masses and/or extranodal lesions having a score of 4 or 5 with no significant change in FDG uptake from baseline at interim or end of treatment, no new lesions, and no change in bone marrow from baseline. Progressive disease (i.e., progressive metabolic disease) is characterized by individual target nodes/nodal masses having a score 4 or 5 with an increase in intensity of uptake from baseline and/or new foci compatible with lymphoma, new FDG-avid foci consistent with lymphoma at interim or end-of-treatment assessment, no non-measured lesions, new FDG-avid foci consistent with lymphoma rather than another etiology (e.g., infection, inflammation), and new or recurrent FDG-avid foci. RECIL 2017 criteria can also be used to asses response based on assessment of target lesions. A complete response is characterized by complete disappearance of all target lesions and all nodes with long axis <10 mm, ≥30% decrease in the sum of longest diameters of target lesions (PR) with normalization of FDG-PET, normalization of FDG-PET (Deauville score 1-3), no involvement of bone marrow, and no new lesions. A partial response is characterized by ≥30% decrease in the sum of longest diameters of target lesions but not a complete response, a positive FDG-PET (Deauville score 4-5), any bone marrow involvement, and no new lesions. A minor response is characterized by ≥10% decrease in the sum of longest diameters of target lesions but not a PR (<30%), any FDG-PET, any bone marrow involvement, and no new lesions. Stable disease is characterized by <10% decrease or ≤20% increase in the sum of longest diameters of target lesions, any FDG-PET, any bone marrow involvement, and no new lesions. Progressive disease is characterized by >20% increase in the sum of longest diameters of target lesions, for small lymph nodes measuring <15 mm post therapy, a minimum absolute increase of 5 mm and the long diameter should exceed 15 mm, appearance of a new lesion, any FDG-PET, any bone marrow involvement, and the appearance of new lesions or no new lesions.

As used herein, the term “relapse” or “relapsed” refers to the recurrence and progression of cancer after a patient had achieved a complete response to prior cancer therapy, or to the progression of cancer in a patient that had achieved a partial response to prior cancer therapy.

As used herein, the term “monoclonal antibody” refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

As used herein, the term “anti-CD52 antibody” refers to an antibody, or antibody fragment or conjugate, having specificity for a CD52 protein expressed on the cell surface of human T cells. In some examples, an anti-CD52 antibody can be a monoclonal antibody. In some cases, an anti-CD52 antibody can be alemtuzumab (i.e., CAMPATH). In some cases, an anti-CD52 antibody can be ALLO-647 (Allogene Therapeutics, San Francisco, CA).

As used herein, the term “anti-CD3 antibody” refers to an antibody, or antibody fragment or conjugate, having specificity for a CD3 protein expressed on the cell surface of human T cells. In some examples, an anti-CD3 antibody can be a monoclonal antibody. In some cases, an anti-CD3 antibody can be muromonab-CD3 (Orthoclone OKT3™) otelixizumab, teplizumab, foralumab, visilizumab, or derivatives thereof which have specificity for CD3.

As used herein, “detectable cell surface expression of CD3” refers to the ability to detect CD3 on the cell surface of a cell (e.g., a genetically-modified cell described herein) using standard experimental methods. Such methods can include, for example, immunostaining and/or flow cytometry specific for CD3. In certain embodiments, the method for determining detectable cell surface expression of CD3 in the disclosed methods is flow cytometry specific for CD3. Methods for detecting cell surface expression of CD3 on a cell include those described in the examples herein, and, for example, those described in MacLeod et al. (2017) Molecular Therapy 25(4): 949-961, the entire disclosure of which is incorporated by reference herein.

As used herein, “detectable cell surface expression of an endogenous alpha/beta TCR” refers to the ability to detect one or more components of the TCR complex (e.g., an alpha/beta TCR complex) on the cell surface of an immune cell (e.g., a CAR T cell), or a population of immune cells (e.g., CAR T cells) described herein, using standard experimental methods. Such methods can include, for example, immunostaining and/or flow cytometry specific for components of the TCR itself, such as a TCR alpha or TCR beta chain, or for components of the assembled cell surface TCR complex, such as CD3. In certain embodiments, the method for determining detectable cell surface expression of an endogenous alpha/beta TCR in the disclosed methods is flow cytometry specific for CD3.

Methods for detecting cell surface expression of an endogenous TCR (e.g., an alpha/beta TCR) on an immune cell include those described in MacLeod et al. (2017) Molecular Therapy 25(4): 949-961.

As used herein, the term “no detectable cell surface expression of CD3” refers to lack of detection of CD3 on the surface of an immune cell (e.g., a CAR T cell) described herein, or population of immune cells (e.g., CAR T cells) described herein, within the limits of detection of standard experimental methods in the art. Methods for detecting cell surface expression of CD3 on an immune cell include those described in MacLeod et al. (2017). This term may embrace, in some examples, no detectable cell surface expression of an endogenous alpha/beta TCR, using one or more standard methods for detecting cell surface expression of an endogenous TCR on an immune cell, as CD3 is a component of the assembled cell surface TCR complex.

As used herein, the term “proliferate in vivo” refers to an expansion in the number of genetically-modified human immune cells described herein in a subject following administration during immunotherapy. Such proliferation or expansion can be determined by methods known in the art and those shown in the examples herein, which include, for example, utilizing PCR analysis to determine the number of copies of a transgene (e.g., a CAR or exogenous TCR transgene) per mg of DNA isolated from peripheral blood mononuclear cells over a time course following administration of the pharmaceutical composition comprising the genetically-modified human immune cells.

As used herein, a “template nucleic acid” or “donor template” refers to a nucleic acid sequence that is desired to be inserted into a cleavage site within a cell's genome. Such template nucleic acids or donor templates can comprise, for example, a transgene, such as an exogenous transgene, which encodes a protein of interest (e.g., a CAR or an exogenous TCR). The template nucleic acid or donor template can comprise 5′ and 3′ homology arms having homology to 5′ and 3′ sequences, respectively, that flank a cleavage site in the genome where insertion of the template is desired. Insertion can be accomplished, for example, by homology-directed repair (HDR).

As used herein, the term “transgene” refers to a nucleic acid molecule that encodes a polypeptide or RNA that is heterologous to the vector sequences flanking the coding sequence or is intended for transfer or has been transferred to a non-native cell or genomic locus.

As used herein, with respect to a protein, the term “recombinant” or “engineered” means having an altered amino acid sequence as a result of the application of genetic engineering techniques to nucleic acids which encode the protein, and cells or organisms which express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In accordance with this definition, a protein having an amino acid sequence identical to a naturally-occurring protein but produced by cloning and expression in a heterologous host, is not considered recombinant.

As used herein, the term “exogenous” or “heterologous” in reference to a nucleotide sequence or amino acid sequence is intended to mean a sequence that is purely synthetic, that originates from a foreign species, or, if from the same species, is substantially modified from its native form in composition and/or genomic locus by deliberate human intervention.

As used herein, the term “endogenous” in reference to a nucleotide sequence or protein is intended to mean a sequence or protein that is naturally comprised within or expressed by a cell.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein with respect to recombinant proteins, the term “modification” means any insertion, deletion, or substitution of an amino acid residue in the recombinant sequence relative to a reference sequence (e.g., a wild-type or a native sequence).

As used herein, the term “inactivation” or “inactivated” or “disrupted” or “disrupts” or “disrupts expression” or “disrupting a target sequence” refers to the introduction of a mutation (e.g., an insertion, a deletion, a substitution, or a frameshift mutation) that interferes with the gene function and prevents expression and/or function of the polypeptide/expression product encoded thereby. For example, nuclease-mediated inactivation or disruption of a gene can result in the expression of a truncated protein and/or expression of a protein that does not retain its wild-type function. Additionally, introduction of a donor template (e.g., a transgene) into a gene can result in no expression of an encoded protein, expression of a truncated protein, and/or expression of a protein that does not retain its wild-type function.

As used herein, the term “a control” or “a control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype.

As used herein with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences which maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

The terms “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, a “vector” or “recombinant DNA vector” may be a construct that includes a replication system and sequences that are capable of transcription and translation of a polypeptide-encoding sequence in a given host cell. If a vector is used then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention.

As used herein, a “vector” can also refer to a viral vector. Viral vectors can include, without limitation, retroviral vectors, lentiviral vectors, adenoviral vectors, and adeno-associated viral vectors (AAV).

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

### 2. Principle of the Invention

The invention is based, in part, on the unexpected observation that administration of an allogeneic cell therapy (i.e., adoptive cellular immunotherapy) regimen is surprisingly effective in a cohort of cancer patients who previously received an autologous cell therapy regimen, had a response, and then relapsed, when compared to patients who were naïve to cell therapy. High overall response rates, and high complete response rates, were observed in this cohort relative to patients who had not previously received an autologous cell therapy. Additionally, analysis of this cohort revealed that an administered lymphodepletion regimen surpassingly produced a deeper and more persistent suppression of the host immune system, which may enable the allogeneic cell therapy to engraft and expand more effectively, thereby enhancing the efficacy of the treatment. Indeed, it was observed that the engineered cells administered in the allogeneic cell therapy expanded to a higher level or to a greater degree, persisted in the patient for a longer period of time, and achieved a higher area under the curve, than the same cells administered to patients who were cell therapy naïve. These findings are potentially valuable for this patient population, who have few therapeutic options following relapse.

### 3. Treatment of Subjects Who Received Prior Autologous Cell Therapy

Described herein are allogeneic cell therapy methods for reducing the number of cancer cells in a subject, which can further be utilized as a method for treating a cancer in the subject (i.e., an immunotherapy). The methods include the administration of a lymphodepletion regimen that comprises one or more chemotherapeutic lymphodepletion agents. Administration of the lymphodepletion regimen prior to the administration of the cell therapy itself is designed to suppress the host immune system and to enhance engraftment and expansion of the cell therapy. The methods further include administering an effective dose of a pharmaceutical composition comprising a population of human immune cells, wherein a plurality of the human immune cells are genetically-modified human immune cells that express an engineered antigen receptor that has specificity for an antigen on the cancer cells. The key aspect of the invention is that the patients in which these methods are practiced have previously received an autologous cell therapy, had a response to that autologous cell therapy, and subsequently relapsed.

Being an allogeneic cell therapy, the human immune cells utilized in the methods are not derived or obtained from the subject, but rather from another healthy subject.

In some examples, the autologous cell therapy previously administered to the subject was directed against the same cancer cell antigen that is targeted in the current methods. For example, if the current method is directed against CD19, the prior autologous cell therapy would also have been directed against CD19. In other examples, the autologous cell therapy was directed against a different antigen than targeted by the current methods. For example, if the current method is directed against CD19, the prior autologous cell therapy may have been directed against another antigen, such as CD20, which is also present on the same target cancer cells.

An aspect of the methods is that the subjects achieved a response to the prior autologous cell therapy. The response may have been a complete response, as determined by a physician with knowledge of that particular cancer. A complete response to therapy might manifest as a complete absence of detectable cancer in the subject. In other examples, the response may have been a partial response, as determined by a physician with knowledge of that particular cancer. A partial response to therapy might manifest as a reduction in the number of cancer cells in the subject, although cancer cells are still detectable. Standards for evaluating a response to therapy for a given cancer, whether a complete response, partial response, or other designation, are known in the art and would be within the skill of a physician to determine.

In particular examples, the subject received a stem cell transplant prior to relapse. In some such examples, the subject received an allogeneic (i.e., donor-derived) stem cell transplant prior to relapse.

The autologous cell therapy previously received by the subject refers to a therapy in which a patient's own immune cells are isolated, modified, expanded, and re-introduced into the same patient in order to achieve a therapeutic outcome, such as the treatment of a disease. The isolated immune cells can be, for example, a patient's own T cells or NK cells. In particular types of autologous cell therapy, the patient's immune cells are modified to express an engineered antigen receptor, such as a CAR or an exogenous TCR which have specificity for an antigen present on the surface of the target cancer cells. Examples of such therapies include the use of a patient's T cells expressing a CAR (i.e., “autologous chimeric antigen receptor (CAR) T cell therapy”). There are a number of approved autologous CAR T cell therapies available to patients, including YESCARTA™, KYMRIAH™, TECARTUS™ BREYANZI®, or ABECMA®, which may be the prior autologous cell therapy received by the present subjects. In another example, the prior autologous cell therapy could include the use of a patient's T cells expressing an exogenous TCR (i.e., “autologous T cell therapy comprising administration of T cells expressing an exogenous TCR”), or the use of a patient's NK cells expressing a CAR (i.e., “autologous CAR natural killer (CAR NK) cell therapy”) or expressing an exogenous TCR (i.e., “autologous NK cell therapy comprising administration of NK cells expressing an exogenous TCR”).

In some embodiments, the present methods are performed within about 15 days after relapse following the autologous cell therapy. In some embodiments, the method is performed within about 30 days after relapse following the autologous cell therapy. In some embodiments, the method is performed within about 45 days after relapse following the autologous cell therapy. In some embodiments, the method is performed within about 60 days after relapse following the autologous cell therapy. In some embodiments, the method is performed within about 75 days after relapse following the autologous cell therapy. In some embodiments, the method is performed within about 90 days after relapse following the autologous cell therapy. In some embodiments, the method is performed within about 120 days after relapse following the autologous cell therapy. In some embodiments, the method is performed within about 150 days after relapse following the autologous cell therapy. In some embodiments, the method is performed within about 180 days after relapse following the autologous cell therapy. In some embodiments, the method is performed within about one year after relapse following the autologous cell therapy. The time at which the present methods are initiated with the subject may vary depending on a number of factors, including the type of cancer and the progression of the cancer after relapse. For example, in cases where the cancer is slower progressing or indolent, a longer period of time may be acceptable before initiating the present methods.

In some embodiments, the subject achieves a response after completion of the present method. In some embodiments, the response is a partial response as determined by a physician with knowledge of that particular cancer. In some embodiments, the response is a complete response as determined by a physician with knowledge of that particular cancer. As previously discussed, standards for evaluating a response to therapy for a given cancer, whether a complete response, partial response, or other designation, are known in the art and would be within the skill of a physician to determine.

The response to the present methods may persist for various lengths of time. In some examples, the response persists for at least 28 days. In some examples, the response persists for about 45 days. In some examples, the response persists for at least 60 days. In some examples, the response persists for about 75 days. In some examples, the response persists for at least 90 days. In some examples, the response persists for about 120 days. In some examples, the response persists for at least 150 days. In some examples, the response persists for at least 180 days. In some examples, the response persists for up to one year or longer.

In some examples of the methods, the response to the present method persists longer than the response to the autologous cell therapy.

In some examples of the methods, the genetically-modified human immune cells expand to a higher level or to a greater degree in the subject compared to a control subject that has not previously received the autologous cell therapy. In some examples, the genetically-modified human immune cells expand to achieve a higher area under the curve (AUC) in the subject compared to a control subject that has not previously received the autologous cell therapy. In some examples, the lymphodepletion regimen reduces the number of endogenous T cells to a lower number in the subject compared to a control subject that has not previously received autologous cell therapy. In some examples, the lymphodepletion regimen reduces the number of endogenous T cells for a longer period of time in the subject compared to a control subject that has not previously received autologous cell therapy.

Various types of cancer are envisioned to be treated by the present methods. In some examples, the cancer is a hematological cancer. In some such examples, the cancer is a cancer of B cell origin or multiple myeloma. In some examples, the cancer of B cell origin is acute lymphoblastic leukemia (ALL), chronic lymphocytic leukemia (CLL), small lymphocytic lymphoma (SLL), or non-Hodgkin lymphoma (NHL). In some examples, the cancer is mantle cell lymphoma (MCL) or diffuse large B cell lymphoma (DLBCL). In other example, the cancer is a solid tumor cancer.

### 4. Lymphodepletion Regimens

The present invention includes methods of reducing the number of target cells (e.g., cancer cells) in a subject. Such methods include administering to the subject a lymphodepletion regimen, for example, prior to administration of a pharmaceutical composition comprising a population of human immune cells, wherein a plurality of such human immune cells are genetically-modified human immune cells, such as T cells or NK cells, that express.

Lymphodepletion regimens of the invention include the administration of one or more chemotherapeutic lymphodepletion agents. Pre-treatment or pre-conditioning patients prior to cell therapies with one or more chemotherapeutic lymphodepletion agents improves the efficacy of the cellular therapy by reducing the number of endogenous host lymphocytes in the subject, thereby providing a more optimal environment for administered cells to proliferate once administered to the subject. An effective dose of one or more chemotherapeutic lymphodepletion agents can result in the reduction of one or more endogenous lymphocytes (e.g., B cells, T cells, and/or NK cells) in the subject by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control; e.g., relative to a starting amount in the subject undergoing treatment, relative to a pre-determined threshold, or relative to an untreated subject.

In some embodiments, 1, 2, 3, 4, or more chemotherapeutic lymphodepletion agents may be included in the lymphodepletion regimen.

Chemotherapeutic lymphodepletion agents can refer to non-biological materials, such as small molecules, that can be administered as part of a lymphodepletion regimen to reduce endogenous lymphocytes in the subject for immunotherapy. In some examples, the chemotherapeutic lymphodepleting agent can be lymphodepleting but non-myeloablative. Chemotherapeutic lymphodepletion agents can include those known in the art including, without limitation, cyclophosphamide, fludarabine, bendamustine, melphalan, 6-mercaptopurine (6-MP), daunorubicin, cytarabine, L-asparaginase, methotrexate, prednisone, dexamethasone, nelarabine, or combinations thereof. In some embodiments, the chemotherapeutic lymphodepletion agent is a nucleoside analog, such as fludarabine. In some embodiments, the chemotherapeutic lymphodepletion agent is an alkylating agent, such as cyclophosphamide. In certain embodiments, the methods herein involve administering a combination of chemotherapeutic lymphodepletion agents, such as a combination of a nucleoside analog (e.g., fludarabine) and an alkylating agent (e.g., cyclophosphamide).

The lymphodepletion regimen administered during the method of the invention can be administered in an amount effective (i.e., an effective dose) to deplete or reduce the quantity of endogenous lymphocytes in the subject, for example, by 1%, 5%, 10%, 20%, 30%, 40%, 50%, 60%, 70%, 80%, 90%, 91%, 92%, 93%, 94%, 95%, 96%, 97%, 98%, 99%, or more, relative to a control, e.g., relative to a starting amount in the subject undergoing treatment, relative to a pre-determined threshold, or relative to an untreated subject, prior to administration of the pharmaceutical composition (e.g., a population of human immune cells). The reduction in lymphocyte count can be monitored using conventional techniques known in the art, such as by flow cytometry analysis of cells expressing characteristic lymphocyte cell surface antigens in a blood sample withdrawn from the subject at varying intervals during treatment with the antibody. According to some embodiments, when the concentration of lymphocytes has reached a minimum value in response to the lymphodepletion regimen, the physician may conclude the lymphodepletion therapy and may begin preparing the subject for administration of the pharmaceutical composition.

In various embodiments, the one or more chemotherapeutic lymphodepletion agents can be administered one day to one month (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days) prior to administration of the pharmaceutical compositions described herein. In some embodiments, a chemotherapeutic lymphodepletion agent is administered to the subject three or more days prior to administration of the pharmaceutical composition. In certain embodiments, administration of a chemotherapeutic lymphodepletion agent ends at least one day, at least two days, or at least three days prior to administration of the pharmaceutical composition.

In some embodiments, a chemotherapeutic lymphodepletion agent can be administered as a single dose per day on each of eight consecutive days, as a single dose per day on each of seven consecutive days, as a single dose per day on each of six consecutive days, as a single dose per day on each of five consecutive days, as a single dose per day on each of four consecutive days, as a single dose per day on each of three consecutive days, as a single dose per day on each of two consecutive days, or as a single dose on one day, prior to administration of the pharmaceutical composition.

In some embodiments, the chemotherapeutic lymphodepletion agent is an alkylating agent, such as cyclophosphamide, which is administered as a single dose per day on each of five consecutive days, as a single dose per day on each of four consecutive days, as a single dose per day on each of three consecutive days, as a single dose per day on each of two consecutive days, or as a single dose on one day, prior to administration of the pharmaceutical composition. In certain embodiments, the alkylating agent (e.g., cyclophosphamide) is administered as one dose per day for three consecutive days or one dose per day for two consecutive days. In certain embodiments, administration of the alkylating agent (e.g., cyclophosphamide) ends at least one to three days prior to administration of the pharmaceutical composition. In certain embodiments, the alkylating agent (e.g., cyclophosphamide) is administered as a single dose on each day beginning five days and ending three days before administration of the pharmaceutical composition. In certain embodiments, the alkylating agent (e.g., cyclophosphamide) is administered as a single dose on each day beginning six days and ending four days before administration of the pharmaceutical composition. In certain embodiments, the alkylating agent (e.g., cyclophosphamide) is administered as a single dose on each day beginning four days and ending two days before administration of the pharmaceutical composition.

In some embodiments, the chemotherapeutic lymphodepletion agent is a nucleoside analog (e.g., fludarabine), which is administered as a single dose per day on each of five consecutive days, as a single dose per day on each of four consecutive days, as a single dose per day on each of three consecutive days, as a single dose per day on each of two consecutive days, or as a single dose on one day, prior to administration of the pharmaceutical composition. In other embodiments, the nucleoside analog (e.g., fludarabine) is administered as one dose per day for five consecutive days, for four consecutive days, for three consecutive days, or for two consecutive days. In certain embodiments, administration of a nucleoside analog (e.g., fludarabine) ends at least one to three days prior to administration of the pharmaceutical composition. In certain embodiments, a nucleoside analog (e.g., fludarabine) is administered as a single dose on each day beginning six days and ending four days before administration of the pharmaceutical composition. In certain embodiments, a nucleoside analog (e.g., fludarabine) is administered as a single dose on each day beginning five days and ending three days before administration of the pharmaceutical composition. In certain embodiments, a nucleoside analog (e.g., fludarabine) is administered as a single dose on each day beginning four days and ending two days before administration of the pharmaceutical composition. In certain embodiments, a nucleoside analog (e.g., fludarabine) is administered as a single dose on each day beginning six days and ending three days before administration of the pharmaceutical composition. In certain embodiments, a nucleoside analog (e.g., fludarabine) is administered as a single dose on each day beginning five days and ending two days before administration of the pharmaceutical composition.

In certain embodiments, the alkylating agent (e.g., cyclophosphamide) is administered to the subject daily starting five days and ending three days prior to administration of the pharmaceutical composition, and a nucleoside analog (e.g., fludarabine) is administered to the subject daily starting five days and ending three days prior to administration of the pharmaceutical composition. In certain embodiments, the alkylating agent (e.g., cyclophosphamide) is administered to the subject daily starting five days and ending three days prior to administration of the pharmaceutical composition, and a nucleoside analog (e.g., fludarabine) is administered to the subject daily starting six days and ending three days prior to administration of the pharmaceutical composition.

In some examples, the lymphodepletion regimen comprises administering the alkylating agent (e.g., cyclophosphamide) at a dose of about 400 to about 1500 mg/m²/day. In some examples, the lymphodepletion regimen comprises administering the alkylating agent (e.g., cyclophosphamide) at a dose of about 500 to about 1000 mg/m²/day. In some examples, the lymphodepletion regimen comprises administering the alkylating agent (e.g., cyclophosphamide) at a dose of about 600 to about 800 mg/m²/day. In some examples, the lymphodepletion regimen comprises administering the alkylating agent (e.g., cyclophosphamide) at a dose of about 500 mg/m²/day. In some examples, the lymphodepletion regimen comprises administering the alkylating agent (e.g., cyclophosphamide) at a dose of about 550 mg/m²/day. In some examples, the lymphodepletion regimen comprises administering the alkylating agent (e.g., cyclophosphamide) at a dose of about 600 mg/m²/day. In some examples, the lymphodepletion regimen comprises administering the alkylating agent (e.g., cyclophosphamide) at a dose of about 650 mg/m²/day. In some examples, the lymphodepletion regimen comprises administering the alkylating agent (e.g., cyclophosphamide) at a dose of about 700 mg/m²/day. In some examples, the lymphodepletion regimen comprises administering the alkylating agent (e.g., cyclophosphamide) at a dose of about 750 mg/m²/day. In some examples, the lymphodepletion regimen comprises administering the alkylating agent (e.g., cyclophosphamide) at a dose of about 800 mg/m²/day. In some examples, the lymphodepletion regimen comprises administering the alkylating agent (e.g., cyclophosphamide) at a dose of about 850 mg/m²/day. In some examples, the lymphodepletion regimen comprises administering the alkylating agent (e.g., cyclophosphamide) at a dose of about 900 mg/m²/day. In some examples, the lymphodepletion regimen comprises administering the alkylating agent (e.g., cyclophosphamide) at a dose of about 950 mg/m²/day. In some examples, the lymphodepletion regimen comprises administering the alkylating agent (e.g., cyclophosphamide) at a dose of about 1000 mg/m²/day.

In some examples, the one or more chemotherapeutic agents comprises a nucleoside analog. In some examples, the nucleoside analog is fludarabine.

In some examples, the lymphodepletion regimen comprises administering the nucleoside analog (e.g., fludarabine) at a dose of about 10 to about 40 mg/m²/day. In some examples, the lymphodepletion regimen comprises administering the nucleoside analog (e.g., fludarabine) at a dose of about 20 to about 40 mg/m²/day. In some examples, the lymphodepletion regimen comprises administering the nucleoside analog (e.g., fludarabine) at a dose of about 30 to about 40 mg/m²/day. In some examples, the lymphodepletion regimen comprises administering the nucleoside analog (e.g., fludarabine) at a dose of about 20 mg/m²/day. In some examples, the lymphodepletion regimen comprises administering the nucleoside analog (e.g., fludarabine) at a dose of about 25 mg/m²/day. In some examples, the lymphodepletion regimen comprises administering the nucleoside analog (e.g., fludarabine) at a dose of about 30 mg/m²/day. In some examples, the lymphodepletion regimen comprises administering the nucleoside analog (e.g., fludarabine) at a dose of about 35 mg/m²/day. In some examples, the lymphodepletion regimen comprises administering the nucleoside analog (e.g., fludarabine) at a dose of about 40 mg/m²/day.

In some examples, the lymphodepletion regimen comprises administering the alkylating agent (e.g., cyclophosphamide) at a dose of about 500 mg/m²/day and the nucleoside analog (e.g., fludarabine) at a dose of about 30 mg/m²/day. In some examples, the lymphodepletion regimen comprises administering the alkylating agent (e.g., cyclophosphamide) at a dose of about 750 mg/m²/day and the nucleoside analog (e.g., fludarabine) at a dose of about 30 mg/m²/day. In some examples, the lymphodepletion regimen comprises administering the alkylating agent (e.g., cyclophosphamide) at a dose of about 1000 mg/m²/day and the nucleoside analog (e.g., fludarabine) at a dose of about 30 mg/m²/day.

In some examples, the lymphodepletion regimen comprises administering the alkylating agent (e.g., cyclophosphamide) at a dose of about 500 mg/m²/day and the nucleoside analog (e.g., fludarabine) at a dose of about 30 mg/m²/day daily, each starting 5 days and ending 3 days prior to administration of the pharmaceutical composition.

In some examples, the lymphodepletion regimen comprises administering the alkylating agent (e.g., cyclophosphamide) at a dose of about 750 mg/m²/day and the nucleoside analog (e.g., fludarabine) at a dose of about 30 mg/m²/day daily, each starting 5 days and ending 3 days prior to administration of the pharmaceutical composition.

In some examples, the lymphodepletion regimen comprises administering the alkylating agent (e.g., cyclophosphamide) at a dose of about 1000 mg/m²/day and the nucleoside analog (e.g., fludarabine) at a dose of about 30 mg/m²/day daily, each starting 5 days and ending 3 days prior to administration of the pharmaceutical composition.

In some examples, the lymphodepletion regimen comprises administering the alkylating agent (e.g., cyclophosphamide) at a dose of about 500 mg/m²/day daily starting 5 days and ending 3 days prior to administration of the pharmaceutical composition, and administering the nucleoside analog (e.g., fludarabine) at a dose of about 30 mg/m²/day daily starting 6 days and ending 3 days prior to administration of the pharmaceutical composition.

In some examples, the lymphodepletion regimen comprises administering the alkylating agent (e.g., cyclophosphamide) at a dose of about 750 mg/m²/day daily starting 5 days and ending 3 days prior to administration of the pharmaceutical composition, and administering the nucleoside analog (e.g., fludarabine) at a dose of about 30 mg/m²/day daily starting 6 days and ending 3 days prior to administration of the pharmaceutical composition.

In some examples, the lymphodepletion regimen comprises administering the alkylating agent (e.g., cyclophosphamide) at a dose of about 1000 mg/m²/day daily starting 5 days and ending 3 days prior to administration of the pharmaceutical composition, and administering the nucleoside analog (e.g., fludarabine) at a dose of about 30 mg/m²/day daily starting 6 days and ending 3 days prior to administration of the pharmaceutical composition.

Methods for administering the chemotherapeutic lymphodepletion agents, and their routes of administration, would be known in the art and to those of ordinary skill.

In some embodiments, the lymphodepletion regimen includes no more than a minimal effective dose, as defined herein, of any biological lymphodepletion agent. In some embodiments, the lymphodepletion regimen does not include administration of any biological lymphodepletion regimen. In some examples, the lymphodepletion regimen does not include administration of any biological lymphodepletion agent in an amount greater than about 0.01 mg/kg, 0.03 mg/kg, 0.05 mg/kg, 0.75 mg/kg, 0.1 mg/kg, 0.15 mg/kg, 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, or about 1.0 mg/kg during the 7 day period preceding administration of a pharmaceutical composition of the invention. Thus, in certain examples, the lymphodepletion regimen does not include administration of any biological lymphodepletion agent in an amount greater than about 0.01 mg/kg during the preceding 7 day period. In certain examples, the lymphodepletion regimen does not include administration of any biological lymphodepletion agent in an amount greater than about 0.03 mg/kg during the preceding 7 day period. In certain examples, the lymphodepletion regimen does not include administration of any biological lymphodepletion agent in an amount greater than about 0.05 mg/kg during the preceding 7 day period. In certain examples, the lymphodepletion regimen does not include administration of any biological lymphodepletion agent in an amount greater than about 0.075 mg/kg during the preceding 7 day period. In certain examples, the lymphodepletion regimen does not include administration of any biological lymphodepletion agent in an amount greater than about 0.1 mg/kg during the preceding 7 day period. In certain examples, the lymphodepletion regimen does not include administration of any biological lymphodepletion agent in an amount greater than about 0.15 mg/kg during the preceding 7 day period. In certain examples, the lymphodepletion regimen does not include administration of any biological lymphodepletion agent in an amount greater than about 0.2 mg/kg during the preceding 7 day period. In certain examples, the lymphodepletion regimen does not include administration of any biological lymphodepletion agent in an amount greater than about 0.3 mg/kg during the preceding 7 day period. In certain examples, the lymphodepletion regimen does not include administration of any biological lymphodepletion agent in an amount greater than about 0.4 mg/kg during the preceding 7 day period. In certain examples, the lymphodepletion regimen does not include administration of any biological lymphodepletion agent in an amount greater than about 0.5 mg/kg during the preceding 7 day period. In certain examples, the lymphodepletion regimen does not include administration of any biological lymphodepletion agent in an amount greater than about 0.6 mg/kg during the preceding 7 day period. In certain examples, the lymphodepletion regimen does not include administration of any biological lymphodepletion agent in an amount greater than about 0.7 mg/kg during the preceding 7 day period. In certain examples, the lymphodepletion regimen does not include administration of any biological lymphodepletion agent in an amount greater than about 0.8 mg/kg during the preceding 7 day period. In certain examples, the lymphodepletion regimen does not include administration of any biological lymphodepletion agent in an amount greater than about 0.9 mg/kg during the preceding 7 day period. In certain examples, the lymphodepletion regimen does not include administration of any biological lymphodepletion agent in an amount greater than about 1.0 mg/kg during the preceding 7 day period.

In other embodiments, the lymphodepletion regimen does include administration of a biological lymphodepletion agent.

A biological lymphodepletion agent can be, for example, any biological material, such an antibody, antibody fragment, antibody conjugate, or the like, that can be administered as part of a lymphodepletion regimen to reduce endogenous lymphocytes in the subject for immunotherapy. Such biological lymphodepletion agents can include, for example, a monoclonal antibody, or a fragment thereof. In some examples, the biological lymphodepletion agent has specificity for a T cell antigen; i.e., an antigen expressed on the cell surface of T cells. Examples of such antigens include, without limitation, CD52 and CD3. In a particular example, the biological lymphodepletion agent is an antibody, such as a monoclonal antibody, having specificity for CD52. Such antibodies can include, for example, alemtuzumab (i.e., CAMPATH), ALLO-647 (Allogene Therapeutics, San Francisco, CA), derivatives thereof which bind CD52, or any other CD52 antibody. In another particular example, the biological lymphodepletion agent is an antibody, such as a monoclonal antibody, having specificity for CD3. In some cases, an anti-CD3 antibody can be muromonab-CD3 (Orthoclone OKT3™), otelixizumab, teplizumab, foralumab, visilizumab, or derivatives thereof which have specificity for CD3.

### 5. Human Immune Cells and Populations

The invention provides pharmaceutical compositions comprising populations of human immune cells, wherein a plurality of the human immune cells are genetically-modified human immune cells; e.g., human immune cells, such as human T cells or NK cells, comprising in their genome a transgene encoding an engineered antigen receptor, such as a CAR or an exogenous TCR, which is expressed on the cell surface of the immune cell.

Human immune cells for use in the invention can be obtained from a number of sources including, for example, peripheral blood mononuclear cells, bone marrow, lymph node tissue, cord blood, thymus tissue, tissue from a site of infection, ascites, pleural effusion, spleen tissue, and tumors. In some embodiments of the present disclosure, immune cells are obtained from a unit of blood collected from a subject using any number of techniques known to the skilled artisan. In one embodiment, cells from the circulating blood of an individual are obtained by apheresis. In some examples, the human immune cells utilized in the present methods are derived from induced pluripotent stem cells (iPSCs).

In some embodiments, the genetically-modified human immune cells disclosed herein express a CAR and/or comprise a transgene encoding a CAR within their genome.

Generally, a CAR of the present disclosure will comprise at least an extracellular domain, a transmembrane domain, and an intracellular domain. In some embodiments, the extracellular domain comprises a target-specific binding element otherwise referred to as an extracellular ligand-binding domain or moiety. In some embodiments, the intracellular domain, or cytoplasmic domain, comprises at least one co-stimulatory domain and one or more signaling domains.

In some embodiments, a CAR useful in the invention comprises an extracellular ligand-binding domain having specificity for a cancer cell antigen (i.e., an antigen expressed on the surface of a cancer cell). The choice of ligand-binding domain depends upon the type and number of ligands that define the surface of a target cell. For example, the ligand-binding domain may be chosen to recognize a ligand that acts as a cell surface marker on target cells associated with a particular disease state. Thus, some examples of cell surface markers that may act as ligands for the ligand-binding domain in a CAR can include those associated cancer cells. In some embodiments, a CAR is engineered to target a cancer-specific antigen of interest by way of engineering a desired ligand-binding moiety that specifically binds to an antigen on a cancer cell. In the context of the present disclosure, “cancer antigen” or “cancer-specific antigen” refer to antigens that are common to specific hyperproliferative disorders such as cancer.

In some embodiments, the extracellular ligand-binding domain of the CAR is specific for any antigen or epitope of interest, particularly any cancer antigen or epitope of interest. Such cancers can include, without limitation, cancers of B cell origin or multiple myeloma. In some examples, the cancer of B cell origin is acute lymphoblastic leukemia (ALL), chronic lymphocytic leukemia (CLL), small lymphocytic lymphoma (SLL), or non-Hodgkin lymphoma (NHL). In some examples, the cancer of B cell origin is mantle cell lymphoma (MCL) or diffuse large B cell lymphoma (DLBCL). As non-limiting examples, in some embodiments the antigen of the target is a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD22, CD30, CD40, CD79B, ILIRAP, glypican 3 (GPC3), CLL-1, disialoganglioside GD2, ductal-epithelial mucine, gp36, TAG-72, glycosphingolipids, glioma-associated antigen, B-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, insulin growth factor (IGFI)-1, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the A1 domain of tenascin-C(TnC Al) and fibroblast associated protein (fap); a lineage-specific or tissue specific antigen such as CD3, CD4, CD8, CD24, CD25, CD33, CD34, CD38, CD123, CD133, CD138, CTLA-4, B7-1 (CD80), B7-2 (CD86), endoglin, a major histocompatibility complex (MHC) molecule, BCMA (CD269, TNFRSF 17), CS1, or a virus-specific surface antigen such as an HIV-specific antigen (such as HIV gp120); an EBV-specific antigen, a CMV-specific antigen, a HPV-specific antigen such as the E6 or E7 oncoproteins, a Lasse Virus-specific antigen, an Influenza Virus-specific antigen, as well as any derivate or variant of these surface markers.

In some examples, the extracellular ligand-binding domain or moiety is an antibody, or antibody fragment. An antibody fragment can, for example, be at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHi domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

In some embodiments, a CAR comprises a transmembrane domain which links the extracellular ligand-binding domain with the intracellular signaling and co-stimulatory domains via a hinge region or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (α chain), p75 (β chain) or γ chain, subunit chain of Fc receptors (e.g., Fcy receptor III) or CD proteins such as the CD8 alpha chain. In certain examples, the transmembrane domain is a CD8 alpha domain. Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcγRllla receptor or IgG1. In certain examples, the hinge region can be a CD8 alpha domain.

Intracellular signaling domains of a CAR are responsible for activation of at least one of the normal effector functions of the cell in which the CAR has been placed and/or activation of proliferative and cell survival pathways. The term “effector function” refers to a specialized function of a cell. Effector function of a T cell, for example, may be cytolytic activity or helper activity including the secretion of cytokines. The intracellular stimulatory domain can include one or more cytoplasmic signaling domains that transmit an activation signal to the T cell following antigen binding. Such cytoplasmic signaling domains can include, without limitation, a CD3 zeta signaling domain.

The intracellular stimulatory domain can also include one or more intracellular co-stimulatory domains that transmit a proliferative and/or cell-survival signal after ligand binding. In some cases, the co-stimulatory domain can comprise one or more TRAF-binding domains. Such intracellular co-stimulatory domains can be any of those known in the art and can include, without limitation, those co-stimulatory domains disclosed in WO 2018/067697 including, for example, Novel 6 (“N6”). Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In a particular embodiment, the co-stimulatory domain is an N6 domain. In another particular embodiment, the co-stimulatory domain is a 4-1BB co-stimulatory domain. In another particular embodiment, the co-stimulatory domain is a CD28 co-stimulatory domain.

In some embodiments, the genetically-modified human immune cells disclosed herein express an exogenous TCR and/or comprise a transgene encoding an exogenous TCR within their genome. As used herein, the terms “exogenous T cell receptor” or “exogenous TCR” refer to a TCR whose sequence is introduced into the genome of an immune cell (e.g., a human T or NK cell) that may or may not endogenously express the TCR. Expression of an exogenous TCR on an immune effector cell can confer specificity for a specific epitope or antigen (e.g., an epitope or antigen preferentially present on the surface of a cancer cell or other disease-causing cell or particle). Such exogenous T cell receptors can comprise alpha and beta chains or, alternatively, may comprise gamma and delta chains. Exogenous TCRs useful in the invention may have specificity to any antigen or epitope of interest. In some examples, exogenous TCRs can include an extracellular ligand-binding domain comprising an antibody, or antibody fragment, having specificity for a target antigen. Such an antibody fragment can be, for example, a single-chain variable fragment (scFv). An “exogenous T cell receptor” or “exogenous TCR” can also refer to a cell surface TCR complex that incorporates one or more genetically-modified and/or exogenous TCR components (e.g., a TRuC; see for example, WO2016187349, WO2018026953, WO2018067993, WO2018098365, WO2018119298, and WO2021035170). Accordingly, in embodiments wherein a nucleic acid sequence encodes an “exogenous T cell receptor” or “exogenous TCR”, this can refer to a sequence encoding one or more genetically-modified and/or exogenous TCR complex components that, when expressed, associate with endogenous TCR components to form a functional modified TCR complex on the cell surface.

As non-limiting examples, in some embodiments the exogenous TCR has specificity for a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD22, CD30, CD40, CD79B, ILIRAP, glypican 3 (GPC3), CLL-1, disialoganglioside GD2, ductal-epithelial mucine, gp36, TAG-72, glycosphingolipids, glioma-associated antigen, B-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, insulin growth factor (IGFI)-1, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the A1 domain of tenascin-C(TnC Al) and fibroblast associated protein (fap); a lineage-specific or tissue specific antigen such as CD3, CD4, CD8, CD24, CD25, CD33, CD34, CD38, CD123, CD133, CD138, CTLA-4, B7-1 (CD80), B7-2 (CD86), endoglin, a major histocompatibility complex (MHC) molecule, BCMA (CD269, TNFRSF 17), CS1, or a virus-specific surface antigen such as an HIV-specific antigen (such as HIV gp120); an EBV-specific antigen, a CMV-specific antigen, a HPV-specific antigen such as the E6 or E7 oncoproteins, a Lasse Virus-specific antigen, an Influenza Virus-specific antigen, as well as any derivate or variant of these surface markers.

Genetically-modified human immune cells of the present invention can comprise an inactivated TCR alpha gene and/or an inactivated TCR beta gene. Inactivation of the TCR alpha gene and/or TCR beta gene occurs in at least one or both alleles where the TCR alpha gene and/or TCR beta gene is being expressed. Thus, inactivation may occur by disruption of one of the alleles of the TCR alpha or TCR beta gene. Accordingly, inactivation of one or both genes prevents expression of the endogenous TCR alpha chain or the endogenous TCR beta chain protein. Expression of these proteins is required for assembly of the endogenous alpha/beta TCR on the cell surface. Thus, inactivation of the TCR alpha gene and/or the TCR beta gene results in genetically-modified immune cells that have no detectable cell surface expression of the endogenous alpha/beta TCR. The endogenous alpha/beta TCR incorporates CD3. Therefore, cells with an inactivated TCR alpha gene and/or TCR beta chain can have no detectable cell surface expression of CD3, e.g., as determined by flow cytometry specific for CD3. In particular embodiments, the inactivated gene is a TCR alpha constant region (TRAC) of the TCR alpha gene.

In some examples, the TCR alpha gene, the TRAC region, or the TCR beta gene is inactivated by insertion of a transgene encoding the engineered antigen receptor (e.g., a CAR or exogenous TCR). In some examples, one or both alleles of the TCR alpha gene is inactivated by insertion of a transgene encoding the engineered antigen receptor (e.g., a CAR or exogenous TCR). In some embodiments, one or both alleles of the TRAC region is inactivated by insertion of a transgene encoding the engineered antigen receptor (e.g., a CAR or exogenous TCR). In some examples, one or both alleles of the TCR beta gene is inactivated by insertion of a transgene encoding the engineered antigen receptor (e.g., a CAR or exogenous TCR). Insertion of the engineered antigen receptor (e.g., a CAR or exogenous TCR) transgene disrupts expression of the endogenous TCR alpha chain or TCR beta chain and, therefore, prevents assembly of an endogenous alpha/beta TCR on the T cell surface. In some examples, the transgene is inserted into the TRAC gene. In a particular example, a transgene is inserted into the TRAC gene within a recognition sequence comprising SEQ ID NO: 1. In particular examples, the transgene is inserted into SEQ ID NO: 1 between nucleotide positions 13 and 14.

As used herein, “detectable cell surface expression of an endogenous alpha/beta TCR” refers to the ability to detect one or more components of the TCR complex (e.g., an alpha/beta TCR complex) on the cell surface of an immune cell using standard experimental methods. Such methods can include, for example, immunostaining and/or flow cytometry specific for components of the TCR itself, such as a TCR alpha or TCR beta chain, or for components of the assembled cell surface TCR complex, such as CD3. Methods for detecting cell surface expression of an endogenous TCR (e.g., an alpha/beta TCR) on an immune cell include those described in the examples herein, and, for example, those described in MacLeod et al. (2017).

Similarly, “detectable cell surface expression of CD3” refers to lack of detection of CD3 on the surface of a T cell (e.g., a CAR T cell) described herein, or population of T cells (e.g., CAR T cells) described herein, as detected using standard experimental methods in the art. Methods for detecting cell surface expression of CD3 on an immune cell include those described in MacLeod et al. (2017).

Human immune cells modified by the present invention, such as human T cells or human NK cells, may require activation prior to introduction of a nuclease and/or an exogenous sequence of interest. For example, T cells can be contacted with anti-CD3 and anti-CD28 antibodies that are soluble or conjugated to a support (e.g., beads) for a period of time sufficient to activate the cells.

Human immune cells of the invention can be further modified to express one or more inducible suicide genes, the induction of which provokes cell death and allows for selective destruction of the cells in vitro or in vivo. In some examples, a suicide gene can encode a cytotoxic polypeptide, a polypeptide that has the ability to convert a non-toxic pro-drug into a cytotoxic drug, and/or a polypeptide that activates a cytotoxic gene pathway within the cell. That is, a suicide gene is a nucleic acid that encodes a product that causes cell death by itself or in the presence of other compounds. A representative example of such a suicide gene is one that encodes thymidine kinase of herpes simplex virus. Additional examples are genes that encode thymidine kinase of varicella zoster virus and the bacterial gene cytosine deaminase that can convert 5-fluorocytosine to the highly toxic compound 5-fluorouracil. Suicide genes also include as non-limiting examples genes that encode caspase-9, caspase-8, or cytosine deaminase. In some examples, caspase-9 can be activated using a specific chemical inducer of dimerization (CID). A suicide gene can also encode a polypeptide that is expressed at the surface of the cell that makes the cells sensitive to therapeutic and/or cytotoxic monoclonal antibodies. In further examples, a suicide gene can encode recombinant antigenic polypeptide comprising an antigenic motif recognized by the anti-CD20 mAb Rituximab and an epitope that allows for selection of cells expressing the suicide gene. See, for example, the RQR8 polypeptide described in WO2013153391, which comprises two Rituximab-binding epitopes and a QBEnd10-binding epitope. For such a gene, Rituximab can be administered to a subject to induce cell depletion when needed. In further examples, a suicide gene may include a QBEnd10-binding epitope expressed in combination with a truncated EGFR polypeptide.

In various embodiments of the invention, the pharmaceutical composition comprises a population of human immune cells. This population of human immune cells includes a plurality of genetically-modified human immune cells expressing a cell surface engineered antigen receptor, such as a CAR or an exogenous TCR. In some examples, the genetically-modified human immune cells represent between about 50% and 80% of the human immune cells in the population. In some examples, the genetically-modified human immune cells represent between about 40% and 75% of the human immune cells in the population. In some examples, the genetically-modified human immune cells represent between about 50% and 75% of the human immune cells in the population. In some examples, the genetically-modified human immune cells represent between about 55% and 75% of the human immune cells in the population. In some examples, the genetically-modified human immune cells represent between about 60% and 75% of the human immune cells in the population.

In some examples of the population of human immune cells, no more than about 0.5% of the cells in the population have detectable cell surface expression of CD3. In certain examples, no more than about 0.3% of the cells in the population have detectable cell surface expression of CD3. In certain examples, no more than about 0.2% of the cells in the population have detectable cell surface expression of CD3. In certain examples, no more than about 0.1% of the cells in the population have detectable cell surface expression of CD3. In certain examples, no cells in the population have detectable cell surface expression of CD3.

In some embodiments, wherein the immune cells are human T cells, the ratio of CD4+ genetically-modified human T cells to CD8+ genetically-modified human T cells in the population is between about 0.3 and about 4.5. In certain embodiments, the ratio of CD4+ genetically-modified human T cells to CD8+ genetically-modified human T cells in the population is between about 0.3 and about 4.2.

T cells which are both CD4+/CCR7+, or both CD8+/CCR7+, can represent T cell populations having a naïve/stem cell memory phenotype, which can be characterized as CD62L+/CD45RA+/CCR7+, and/or a central memory phenotype, which can be characterized as CD62L-/CD45RO+/CCR7+. In some embodiments, the percentage of CD4+ genetically-modified human T cells in the population that are also CCR7+ is between about 20% to about 70%. In certain embodiments, the percentage of CD4+ genetically-modified human T cells in the population that are also CCR7+ is between about 23% to about 65%. In certain embodiments, the percentage of CD4+ genetically-modified human T cells in the population that are also CCR7+ is between about 23% to about 60%.

In some embodiments, the percentage of CD8+ genetically-modified human T cells in the population that are also CCR7+ is between about 10% and about 70%. In some embodiments, the percentage of CD8+ genetically-modified human T cells in the population that are also CCR7+ is between about 10% and about 65%. In certain embodiments, the percentage of CD8+ genetically-modified human T cells in the population that are also CCR7+ is between about 14% and about 60%.

### 6. Pharmaceutical Compositions

The method of the invention provides a pharmaceutical composition comprising a population of human immune cells, including a plurality of genetically-modified human immune cells. Such pharmaceutical compositions can be prepared in accordance with known techniques. See, e.g., Remington, The Science and Practice of Pharmacy (21st ed. 2005). In the manufacture of a pharmaceutical formulation according to the invention, cells are typically admixed with a pharmaceutically acceptable carrier and the resulting composition is administered to a subject. The carrier must, of course, be acceptable in the sense of being compatible with any other ingredients in the formulation and must not be deleterious to the subject. In some embodiments, pharmaceutical compositions of the invention can further comprise one or more additional agents useful in the treatment of a disease in the subject. In additional embodiments, pharmaceutical compositions of the invention can further include biological molecules, such as cytokines (e.g., IL-2, IL-7, IL-15, and/or IL-21), which promote in vivo cell proliferation and engraftment of genetically-modified human immune cells. Pharmaceutical compositions comprising genetically-modified immune cells of the invention can be administered in the same composition as an additional agent or biological molecule or, alternatively, can be co-administered in separate compositions.

The present disclosure also provides populations of human immune cells, comprising a plurality of genetically-modified human immune cells, described herein for use as a medicament. The present disclosure further provides the use of populations of human immune cells, comprising a plurality of genetically-modified human immune cells, described herein in the manufacture of a medicament for treating a disease in a subject in need thereof. In one such aspect, the medicament is useful for cancer immunotherapy in subjects in need thereof.

### 7. Methods of Administering Populations of Human Immune Cells

The method of the invention comprises administering to a subject a pharmaceutical composition comprising a population of human immune cells, wherein the population comprises a plurality of genetically-modified human immune cells, such as genetically-modified human T cells or NK cells expressing an engineered antigen receptor (e.g., a CAR or exogenous TCR). For example, the pharmaceutical composition administered to the subject can comprise an effective dose of genetically-modified human immune cells to reduce the number of target cells (e.g., cancer cells) in the subject, or to treat a cancer in the subject. The administered genetically-modified human immune cells are able to reduce the proliferation, reduce the number, or kill target cells in the recipient.

Unlike antibody therapies, genetically-modified human immune cells of the present disclosure are able to replicate and expand in vivo, resulting in long-term persistence that can lead to sustained control of a disease.

The subject may be administered the pharmaceutical composition, for example, at a dosage of from about 3×10⁴ to about 1×10⁷ genetically-modified human immune cells (e.g., CAR T cells)/kg. In some embodiments, the subject is administered a pharmaceutical composition described herein at a dosage of about 1×10⁵ to about 1×10⁷ genetically-modified human immune cells (e.g., CAR T cells)/kg. In some embodiments, the subject is administered a pharmaceutical composition described herein at a dosage of about 3×10⁵ to about 1×10⁷ genetically-modified human immune cells (e.g., CAR T cells)/kg. In some embodiments, the subject is administered a pharmaceutical composition described herein at a dosage of about 3×10⁴ to about 6×10⁶ genetically-modified human immune cells (e.g., CAR T cells)/kg. In some embodiments, the subject is administered a pharmaceutical composition described herein at a dosage of about 3×10⁵ to about 6×10⁶ genetically-modified human immune cells (e.g., CAR T cells)/kg. In some embodiments, the subject is administered a pharmaceutical composition described herein at a dosage of about 3×10⁵ to about 3×10⁶ genetically-modified human immune cells (e.g., CAR T cells)/kg. In some embodiments, the subject is administered a pharmaceutical composition described herein at a dosage of about 3×10⁴ genetically-modified human immune cells (e.g., CAR T cells)/kg. In some embodiments, the subject is administered a pharmaceutical composition described herein at a dosage of about 3×10⁵ genetically-modified human immune cells (e.g., CAR T cells)/kg. In some embodiments, the subject is administered a pharmaceutical composition described herein at a dosage of about 5×10⁵ genetically-modified human immune cells (e.g., CAR T cells)/kg. In some embodiments, the subject is administered a pharmaceutical composition described herein at a dosage of about 1×10⁶ genetically-modified human immune cells (e.g., CAR T cells)/kg. In some embodiments, the subject is administered a pharmaceutical composition described herein at a dosage of about 1.5×10⁶ genetically-modified human immune cells (e.g., CAR T cells)/kg. In some embodiments, the subject is administered a pharmaceutical composition described herein at a dosage of about 2×10⁶ genetically-modified human immune cells (e.g., CAR T cells)/kg. In some embodiments, the subject is administered a pharmaceutical composition described herein at a dosage of about 2.5×10⁶ genetically-modified human immune cells (e.g., CAR T cells)/kg. In some embodiments, the subject is administered a pharmaceutical composition described herein at a dosage of about 3×10⁶ genetically-modified human immune cells (e.g., CAR T cells)/kg. In some embodiments, the subject is administered a pharmaceutical composition described herein at a dosage of about 3.5×10⁶ genetically-modified human immune cells (e.g., CAR T cells)/kg.

In some embodiments, the subject is administered a pharmaceutical composition described herein at a dosage of about 4×10⁶ genetically-modified human immune cells (e.g., CAR T cells)/kg. In some embodiments, the subject is administered a pharmaceutical composition described herein at a dosage of about 4.5×10⁶ genetically-modified human immune cells (e.g., CAR T cells)/kg. In some embodiments, the subject is administered a pharmaceutical composition described herein at a dosage of about 5×10⁶ genetically-modified human immune cells (e.g., CAR T cells)/kg. In some embodiments, the subject is administered a pharmaceutical composition described herein at a dosage of about 5.5×10⁶ genetically-modified human immune cells (e.g., CAR T cells)/kg. In some embodiments, the subject is administered a pharmaceutical composition described herein at a dosage of about 6×10⁶ genetically-modified human immune cells (e.g., CAR T cells)/kg. In some embodiments, the subject is administered a pharmaceutical composition described herein at a dose of 200×10⁶ genetically-modified human immune cells (e.g., CAR T cells). In some embodiments, the subject is administered a pharmaceutical composition described herein at a dose of 300×10⁶ genetically-modified human immune cells (e.g., CAR T cells). In some embodiments, the subject is administered a pharmaceutical composition described herein at a dose of 400×10⁶ genetically-modified human immune cells (e.g., CAR T cells). In some embodiments, the subject is administered a pharmaceutical composition described herein at a dose of 500×10⁶ genetically-modified human immune cells (e.g., CAR T cells). In some embodiments, the subject is administered a pharmaceutical composition described herein at a dose of 600×10⁶ genetically-modified human immune cells (e.g., CAR T cells). In some embodiments, the subject is administered a pharmaceutical composition described herein at a dose of 700×10⁶ genetically-modified human immune cells (e.g., CAR T cells). In some embodiments, the subject is administered a pharmaceutical composition described herein at a dose of 800×10⁶ genetically-modified human immune cells (e.g., CAR T cells). In some embodiments, the subject is administered a pharmaceutical composition described herein at a dose of 900×10⁶ genetically-modified human immune cells (e.g., CAR T cells). In some embodiments, the subject is administered a pharmaceutical composition described herein at a dose of 1×10⁷ genetically-modified human immune cells (e.g., CAR T cells).

The subject may be administered a first dose of the pharmaceutical composition and a second dose of the pharmaceutical composition. In some cases, the second dose of the pharmaceutical composition is administered without re-administration of the lymphodepletion regimen; i.e., a first and second dose of the pharmaceutical composition is administered following a single lymphodepletion regimen. In some examples, the second dose of the pharmaceutical composition is administered 1, 2, 3, 4, 5, 6, 7, 8, 9, 10, 11, 12, 13, 14, 15, 16, 17, 18, 19, 20, or 21 days following administration of the first dose of the pharmaceutical composition. In certain examples, the second dose of the pharmaceutical composition is administered 10 days following administration of the first dose of the pharmaceutical composition. In some examples, the second dose of the pharmaceutical composition is administered at the same dose of genetically-modified human immune cells/kg as administered in the first dose of the pharmaceutical composition. In other examples, the second dose of the pharmaceutical composition is administered at a different dose of genetically-modified human immune cells/kg than administered in the first dose of the pharmaceutical composition.

In certain examples of the methods, the subject is re-administered both the lymphodepletion regimen and the pharmaceutical composition. In some examples, re-administration of the lymphodepletion regimen and the pharmaceutical composition occurs following a partial response or complete response to the first lymphodepletion regimen and pharmaceutical composition with subsequent progressive disease. In some examples, re-administration of the lymphodepletion regimen and the pharmaceutical composition occurs following no response to the first lymphodepletion regimen and pharmaceutical composition and subsequent progressive disease. In some examples, re-administration of the lymphodepletion regimen and the pharmaceutical composition occurs in subjects having a cancer that remains positive for the cancer cell antigen targeted by the genetically-modified human immune cells. In some examples, re-administration of the lymphodepletion regimen and the pharmaceutical composition occurs about 2 weeks, 4 weeks, 6, weeks, 8 weeks, 10 weeks, 12, weeks, 14, weeks, 16 weeks, 18 weeks, 20 weeks, 22 weeks, 24 weeks, or more after the first administration of the lymphodepletion regimen and pharmaceutical composition. In some examples, the lymphodepletion regimen is re-administered at the same doses and/or schedule as the first administration. In some examples, the lymphodepletion regimen is re-administered at different doses and/or a different schedule as the first administration. In some examples, the lymphodepletion regimen is re-administered according to any of the doses and/or dosing schedules described herein for administration of the first lymphodepletion regimen. In some examples, the pharmaceutical composition is re-administered at the same doses and/or schedule as the first administration. In some examples, the pharmaceutical composition is re-administered at different doses and/or a different schedule as the first administration. In certain examples, the pharmaceutical composition is re-administered at a higher dose than the first administration.

When an “effective amount” or “therapeutic amount” is indicated, the precise amount to be administered can be determined by a physician with consideration of individual differences in age, weight, tumor size (if present), extent of infection or metastasis, and condition of the patient (subject). In some embodiments, a pharmaceutical composition comprising the genetically-modified immune cells or populations thereof described herein is administered at a dosage of 10⁴ to 10⁹ cells/kg body weight, including all integer values within those ranges. In further embodiments, the dosage is 10⁵ to 10⁶ cells/kg body weight, including all integer values within those ranges. In some embodiments, cell compositions are administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

Examples of possible routes of administration of compositions comprising genetically-modified cells or lymphodepletion regimens described herein include parenteral, (e.g., intravenous (IV), intramuscular (IM), intradermal, subcutaneous (SC), or infusion) administration. Moreover, the administration may be by continuous infusion or by single or multiple boluses. In specific embodiments, one or both of the agents is infused over a period of less than about 12 hours, less than about 10 hours, less than about 8 hours, less than about 6 hours, less than about 4 hours, less than about 3 hours, less than about 2 hours, or less than about 1 hour. In still other embodiments, the infusion occurs slowly at first and then is increased over time.

In some embodiments, a pharmaceutical composition of the present disclosure, which comprises genetically-modified human immune cells, targets a cancer cell antigen (i.e., an antigen expressed on the surface of a cancer cell) for the purposes of treating cancer.

In some embodiments, administration of the pharmaceutical compositions of the present disclosure, reduce at least one symptom of a cancer. Symptoms of cancers are well known in the art and can be determined by known techniques.

In some of these embodiments wherein cancer is treated, the subject can be further administered an additional therapeutic agent or treatment, including, but not limited to gene therapy, radiation, surgery, or a chemotherapeutic agent(s) (i.e., chemotherapy).

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Allogeneic CD19 CAR T Therapy in Non-Hodgkin Lymphoma and B-Cell Acute Lymphoblastic Leukemia

PBCAR0191 is an off-the-shelf allogeneic CD19-targeted chimeric antigen receptor (CAR) T cell product derived from qualified donor T cells that have been genetically edited to remove the expression of the endogenous T cell receptor (TCR) and insert the CAR in the same locus. The CAR expressed by PBCAR0191 CAR T cells includes an scFv comprising the VH and VL domains of the FMC63 anti-CD19 antibody, CD8 hinge and transmembrane domains, an intracellular N6 co-stimulatory domain, and an intracellular CD3 zeta signaling domain. The transgene encoding the CAR, which is inserted into the TRAC locus, further includes a JeT promoter that is operatively-lined to the transgene to drive CAR expression. The goal is to achieve anti-tumor effect and reduce the possibility of graft-versus-host disease (GvHD) when it is administered to human leukocyte antigen (HLA)-mismatched patients with CD19 expressing B-cell malignancies.

This is a Phase 1/2a, nonrandomized, open-label, parallel assignment, single-dose, dose-escalation, and dose-expansion study to evaluate the safety and clinical activity of PBCAR0191 in adults with r/r B-ALL (Cohort A) and in adults with r/r B-cell NHL (Cohort N).

This is a multicenter, nonrandomized, open-label, parallel assignment, single-dose, dose-escalation, and dose-expansion study to evaluate the safety and tolerability, find an appropriate dose to optimize safety and efficacy, and evaluate clinical activity of PBCAR0191 in subjects with r/r B-ALL and r/r/NHL. Before initiating PBCAR0191, subjects will be administered lymphodepletion chemotherapy composed of fludarabine and cyclophosphamide. At Day 0 of the Treatment Period, subjects will receive a single intravenous (IV) infusion of PBCAR0191. All subjects are monitored during the treatment period through Day 28. All subjects who receive a dose of PBCAR0191 will be followed in a separate long-term follow-up (LTFU) study for 15 years after exiting this study.

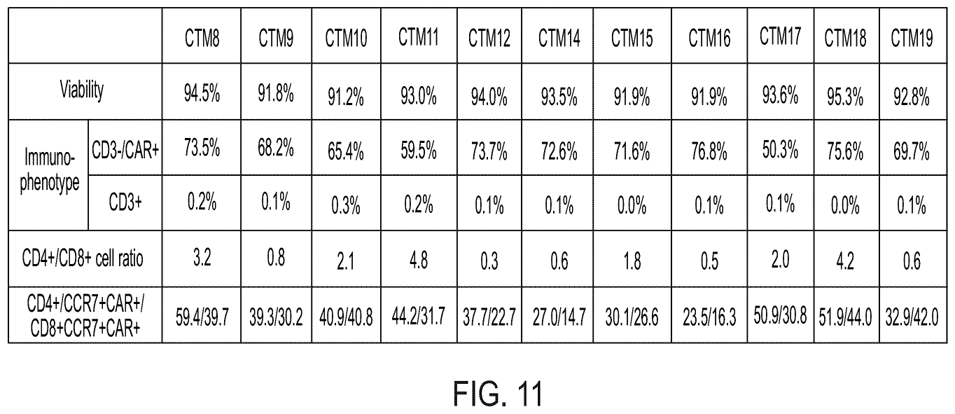

NHL and B-ALL subjects will be enrolled and treated with a single dose of PBCAR0191. The initial dose of PBCAR0191 will be the dose level 4b, which is a 500×10⁶ CAR T cells flat dose, administered on Day 0. Additional doses evaluated include dose level 3 shown below. Clinical trial material is described in FIG. 11 as CTM8 through CTM19.
Dose Level 3 (DL3)=3.0×10⁶/kg (˜1.8-2.5×10⁸ total cells, maximum cell dose is capped at 3.0×10⁸ total cells)
Dose Level 4b (DL4b)=500×10⁶ flat dose

The lymphodepletion regimen includes the administration of cyclophosphamide (750 mg/m²/d) on days −5 to −3 and fludarabine (30 mg/m²/d) on days −6 to −3 prior to PBCAR0191 infusion. This regimen is referred to as a modified lymphodepletion regimen (mLD). An additional lymphodepletion regimen includes the administration of cyclophosphamide (1000 mg/m²/d) on days −5 to −3 and fludarabine (30 mg/m²/d) on days −6 to −3 prior to PBCAR0191. This regimen is referred to as an enhanced lymphodepletion regimen. Potential lymphodepletion regimens include the administration of cyclophosphamide (500 mg/m²/d) on days −5 to −3 and fludarabine (30 mg/m²/d) on days −5 to −3 prior to PBCAR0191. This regimen is referred to as a standard lymphodepletion regimen (sLD).

Primary outcome measures include:
Other Maximum Tolerated Doses (MTD) [Time Frame: Day 1-Day 28] can be determined to determine the maximum tolerated dose (MTD), which is defined as the dose level at which fewer than 33% of patients experience a dose limiting toxicity (DLT) using a 3+3 strategy.
Number of Participants with Dose Limiting Toxicity(ies) [Time Frame: 1 year]
To assess adverse events as dose limiting toxicities as defined by the protocol and CTCAE v5.0.
Objective Response Rate of Patients [Time Frame: 1 year]
To assess clinical activity as response in B-ALL by the NCCN Guidelines on ALL (NCCN, 2017) and in NHL by the revised Lugano Classification (Cheson et al., 2016), both reported as objective response rate.
Area Under the Curve [AUC][Time Frame: Up to 1 year]
To evaluate Area Under the Curve [AUC] of PBCAR0191 in patients tested.

Primary Endpoints: The primary objective of this Phase 1 portion of the ongoing Phase 1/2a trial is to evaluate safety as measured by the occurrence of dose limiting toxicities (DLTs).

Secondary Endpoint: Secondary objectives include assessment of objective tumor responses using standard criteria, and further evaluation of Aes and adverse events of special interest, GvHD, CRS, and ICANS.

### Exploratory Objectives/Endpoints:

Cell expansion and persistence using
   Flow cytometry
   PCR from lysed PBMC (peripheral blood mononuclear cells)
Peripheral cytokine analysis from serum
   Ages eligible for study: 18 Years and older (Adult, Older Adult)
   Sexes eligible for study: All
   Accepts healthy volunteers: No

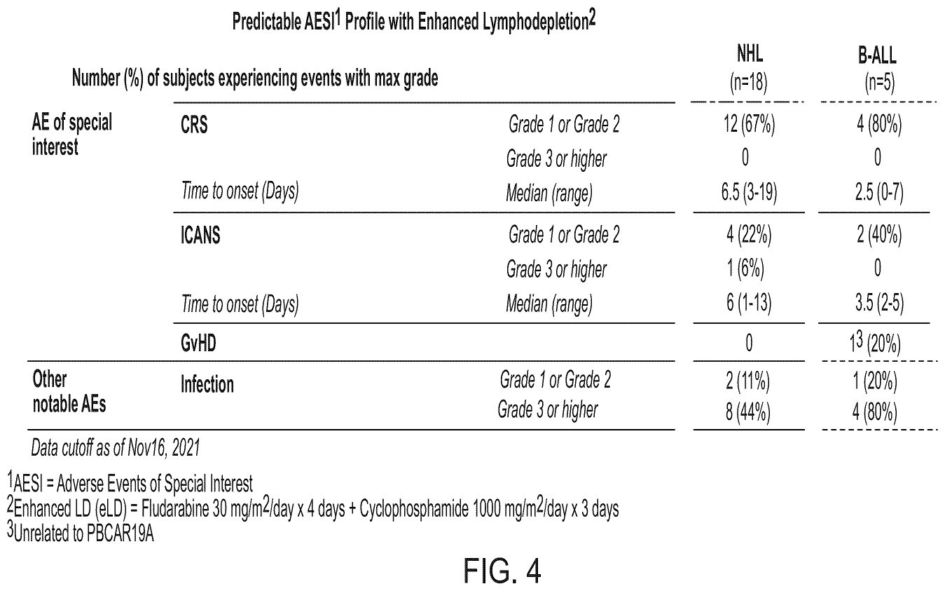

### Key Inclusion Criteria (See Also, FIG. 4)

### Criteria for B-ALL:

Relapsed or refractory CD19+B-cell acute lymphoblastic leukemia (B-ALL).
Philadelphia chromosome positive (Ph+) disease can be eligible if they are intolerant to tyrosine kinase inhibitor (TKI) therapy or if they have relapsed/refractory disease.

### Criteria for NHL:

r/r unequivocally CD19+B-cell NHL that is histologically confirmed by archived tumor biopsy tissue from last relapse and corresponding pathology report. The following types of lymphoma are included:
   Diffuse large B-cell lymphoma (DLBCL) including Richter's transformation
   Primary mediastinal B-cell lymphoma (PMBL)
   FL including Grade 3B or transformed FL
   High-grade B-cell lymphoma
   Mantle cell lymphoma (MCL)
Received at least 2 prior chemotherapy-containing regimens, relapsed after CD19-directed autologous-CAR T cell therapy at Day 28 assessment or later before documented disease progression
Subjects must not have received more than two lines of therapy after auto-CAR T.
Subjects must not have received other CD19 directed therapy, excluding autologous CAR T, within 90 days of anticipated lymphodepletion start date
Measurable or detectable disease according to the Lugano Classification.

### Criteria for Both B-ALL and NHL:

Subject has been treated with 7 or fewer prior lines of therapy overall.
Eastern Cooperative Oncology Group performance status score of 0 or 1.
An estimated life expectancy of at least 12 weeks according to the investigator's judgment.
Seronegative for human immunodeficiency virus antibody (e.g., intact immune function).
Subject has adequate bone marrow, renal, hepatic, pulmonary, and cardiac function defined as:
   Estimated glomerular filtration rate (eGFR) >50 mL/min/1.73 m².
   Alanine aminotransferase (ALT) and aspartate aminotransferase (AST) levels both ≤3 times of upper limit of normal, unless there is suspected disease in the liver.
   Total bilirubin <2.0 mg/dL, except in subjects with Gilbert's syndrome.
   Platelet count ≥50,000/L and ANC of ≥1000/μL. Platelet transfusions within 14 days of screening are not allowed, except for patients in B-ALL or NHL cohorts with high marrow disease burden (>70%) in which case it must be discussed with medical monitor. In the case of extensive bone marrow disease burden, adequate bone marrow recovery after prior treatment is required to be documented.
   Left ventricular ejection fraction >45% as assessed by echocardiogram (ECHO) or multiple gated acquisition scan performed within 1 month before starting lymphodepleting chemotherapy. ECHO results performed within 6 months before Screening and at least 28 days after the last cancer treatment may be acceptable if the subject has not received any treatment with cardiotoxicity risks.
   No clinically significant evidence of pericardial effusion or pleural effusion.
   Baseline oxygen saturation >92% on room air.

### Key Exclusion Criteria

### Criteria for B-ALL:

Burkitt cell (L3 ALL) or mixed-lineage acute leukemia.
Active CNS leukemia.

### Criteria for NHL:

Active hemolytic anemia.
Active CNS lymphoma.
Requirement for urgent therapy due to mass effects such as bowel obstruction, spinal cord, or blood vessel compression.
Any history of CNS disease. If active CNS involvement is suspected, a negative computed tomography (CT)/magnetic resonance imaging (MRI) is required at screening.

### Criteria for all Subjects:

Subject has had a malignancy, besides the malignancies of inclusion (B-cell NHL or CLL/SLL), that in the investigator's opinion, has a high risk of relapse in the next 2 years.
Active uncontrolled fungal, bacterial, viral, protozoal, or other infection. Recent clinically significant fungal, bacterial, viral, protozoal, or other infection must be resolved and not requiring therapeutic anti-microbial medications at least 7 days prior to lymphodepletion. Subjects with elevated CRP must undergo infectious disease (ID) workup and the recommendations discussed with medical monitor to be considered on an individual basis. The CRP must be trending toward the normal range for the laboratory. Simple urinary tract infection and uncomplicated bacterial pharyngitis are permitted if the subject is responding to active treatment and with medical monitor approval.
Any form of primary immunodeficiency (e.g., severe combined immunodeficiency disease).
History of human immunodeficiency virus (HIV) infection.
Active hepatitis B or hepatitis C confirmed by polymerase chain reaction (PCR). Subjects testing positive for inactive hepatitis B will be allowed to enroll if on prophylactic treatment.
Any known uncontrolled cardiovascular disease at the time of Screening that, in the investigator's opinion, is clinically significant and renders the subject ineligible.
History of hypertension crisis or hypertensive encephalopathy within 3 months prior to Screening.
History of severe immediate hypersensitivity reaction to any of the agents used in this study.
Presence of a neurological disorder that, in the opinion of the investigator, may impair the ability to evaluate neurotoxicity.
Abnormal findings during the Screening Period or any other medical condition(s) or laboratory findings that, in the opinion of the investigator, might jeopardize the subject's safety.
History of a genetic syndrome such as Fanconi anemia, Kostmann syndrome, Shwachman-Diamond syndrome, or any other known bone marrow failure syndrome.

### Prior/Concomitant Therapy

Subjects who have received ASCT within 45 days of Screening are excluded.
Subjects who have received systemic corticosteroid (greater than physiologic replacement) therapy for at least 7 days prior to initiating lymphodepletion chemotherapy are excluded.
Subjects who have received a live vaccine within 4 weeks before Screening are excluded. Non-live virus vaccines are not excluded.
Subjects undergoing Radiotherapy to target lesions within 4 weeks before Screening are excluded.
A washout period must be observed for subjects who have received prior treatment for disease under study without documented progression post treatment: 28 days for biologics and 10 days for other systemic anticancer therapy. Any Aes caused by prior treatments must be resolved to Grade 2 or less in severity.
Subject has received other CD19 directed therapy within 90 days of anticipated Lymphodepletion start date.
Subjects who have an indwelling catheter who based on investigator's assessment are at high risk of infection (i.e., pleural, peritoneal, pericardial as well as biliary and ureteral stents) are excluded. This does not apply to intravenous lines.

### Other Exclusions

Pregnant or breastfeeding women.
In the investigator's judgment, the subject is unlikely to complete all protocol required study visits or procedures, including follow-up visits, or is unlikely to comply with the study requirements for participation.
Any mental condition rendering the subject unable to understand the nature, scope, and possible consequences of the study, and/or evidence of an uncooperative attitude.
Additional criteria apply

### Example 2

### Interim Results of PBCAR0191 Study Including Treatment of Patients Who Previously Received Autologous CD19-Directed CAR T Therapy

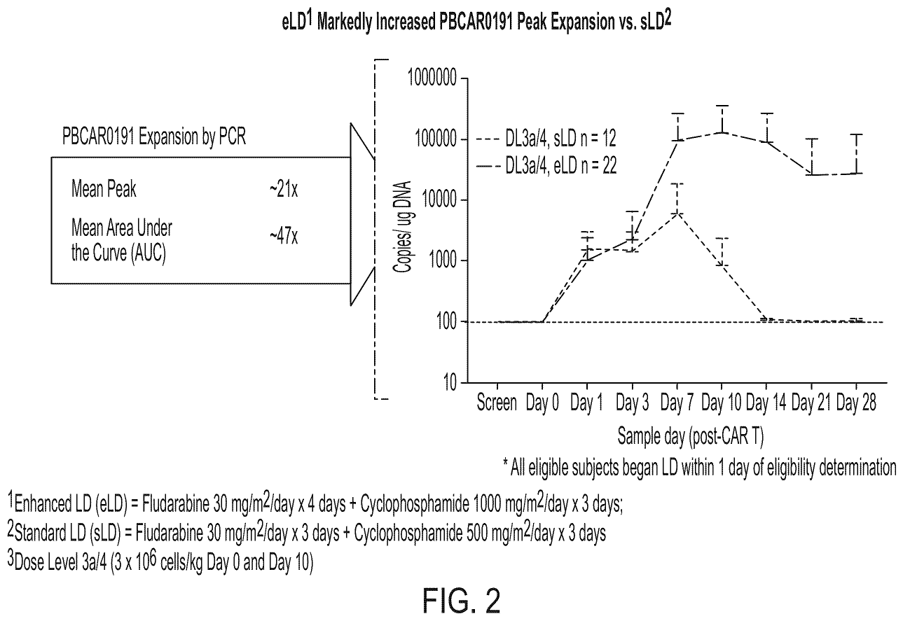

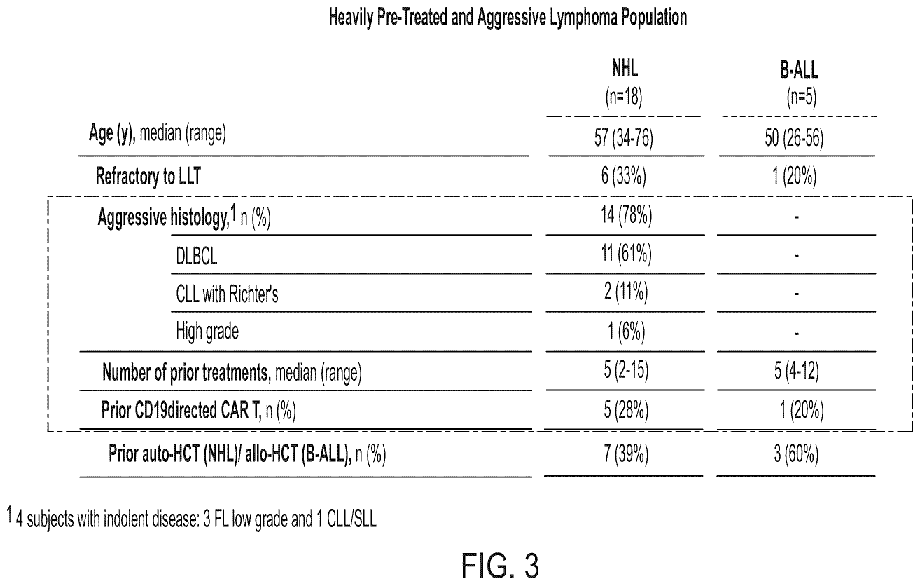

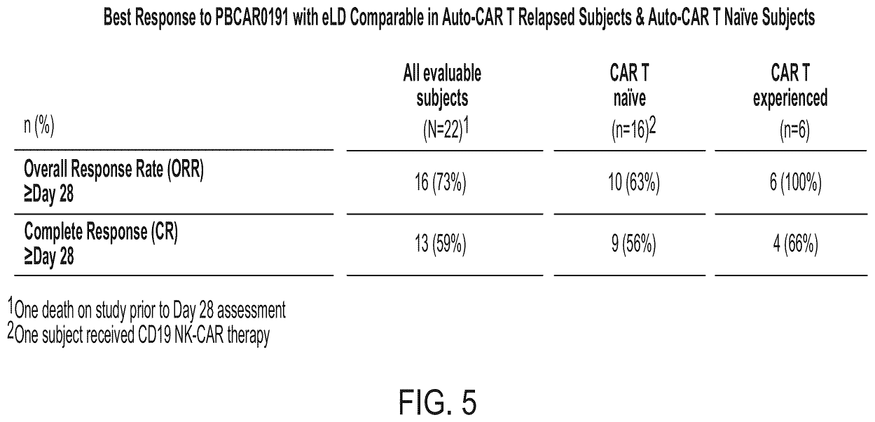

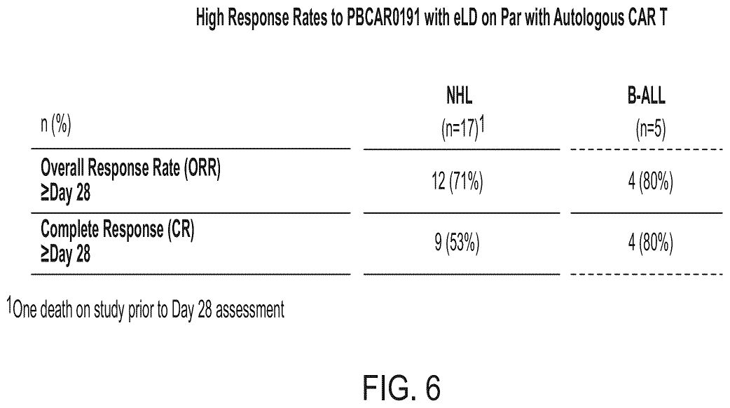

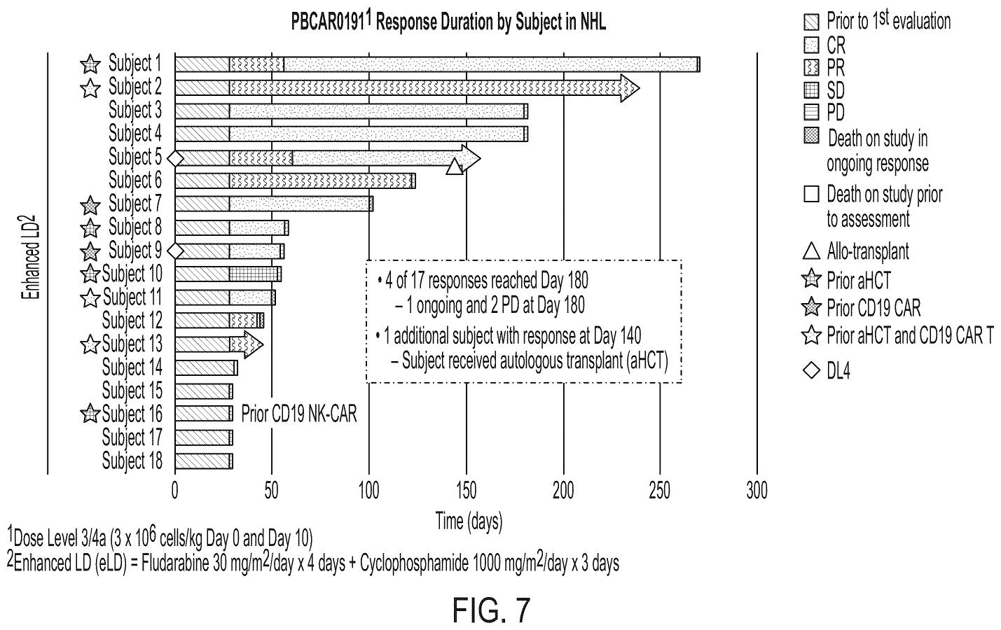

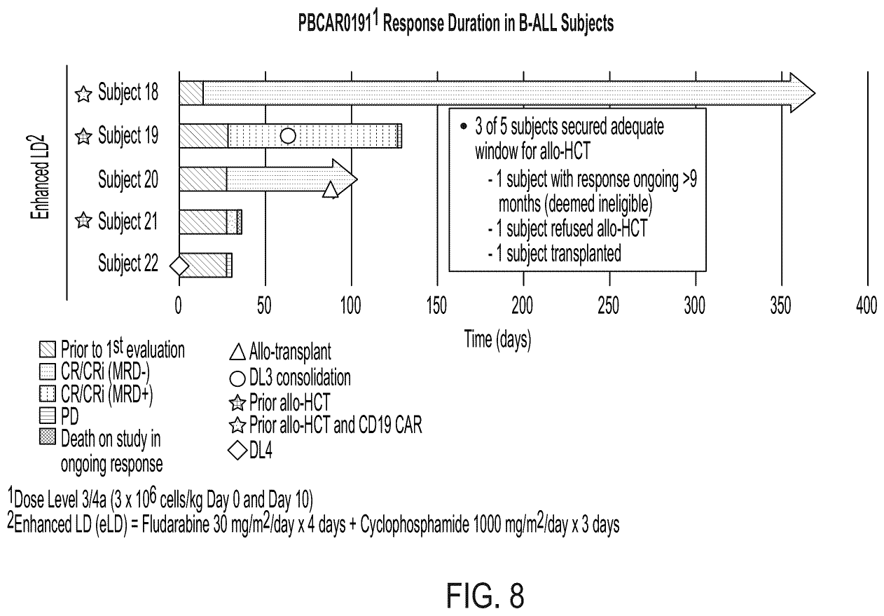

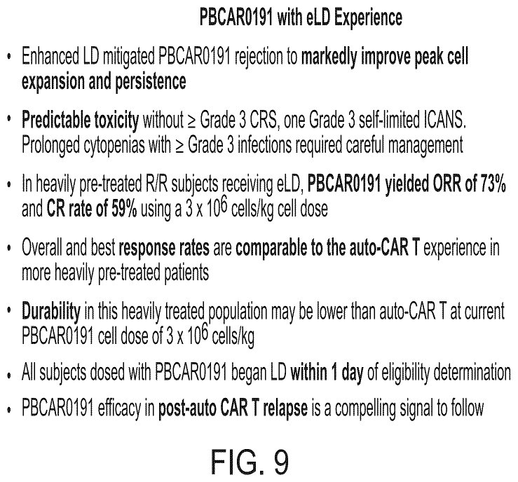

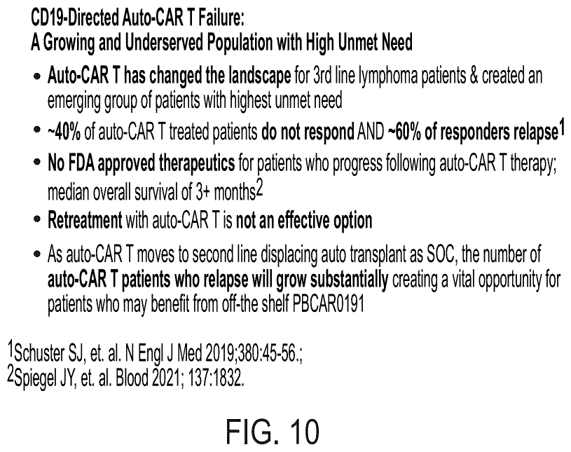

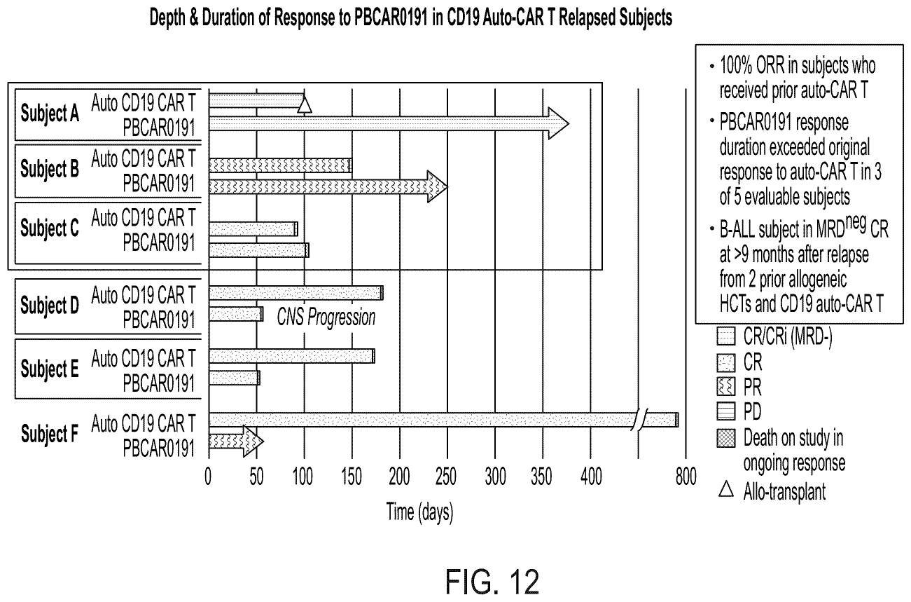

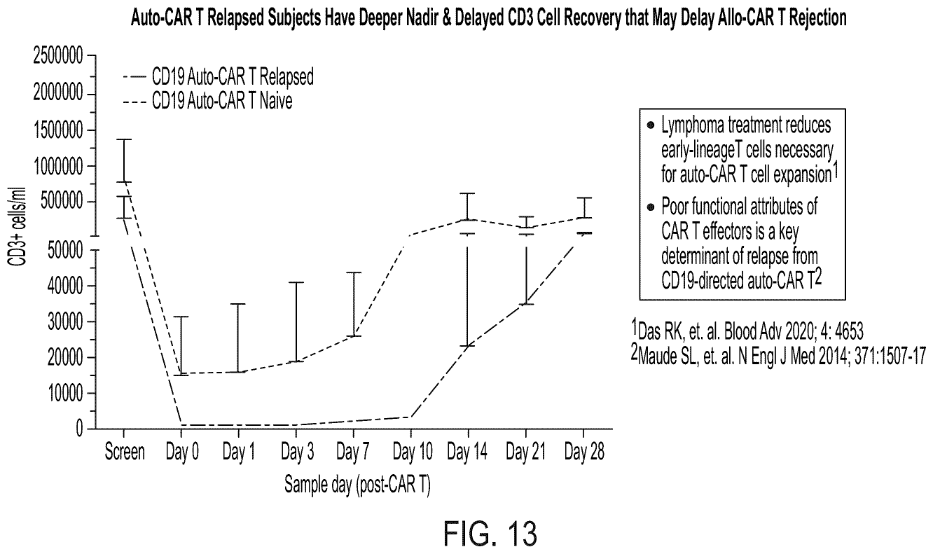

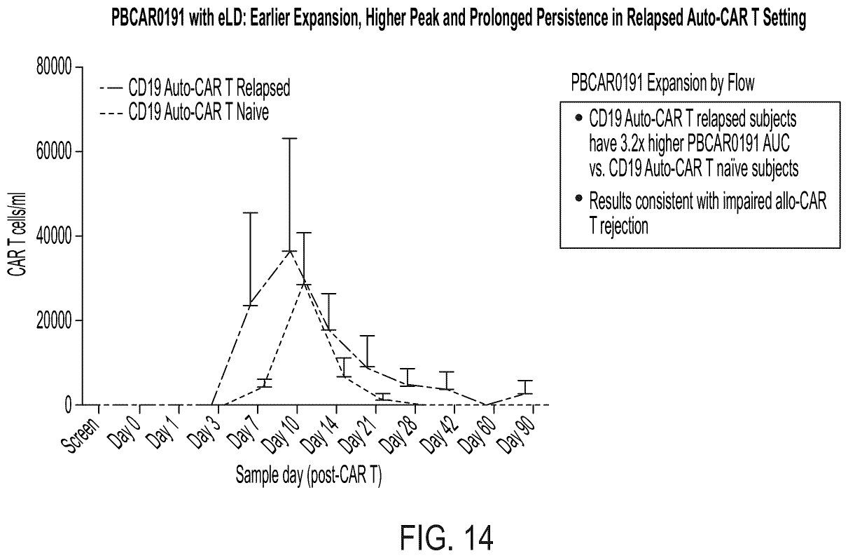

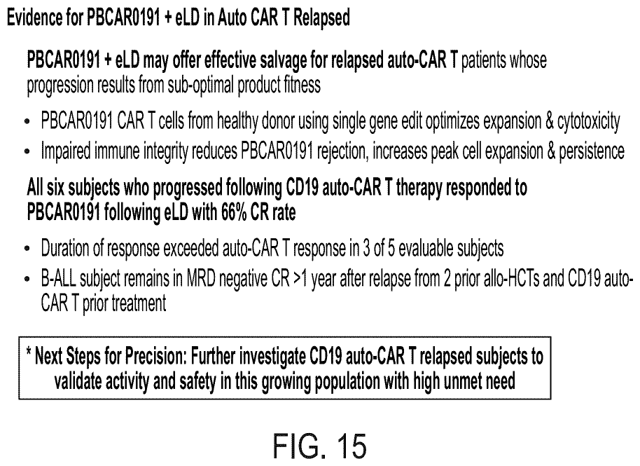

Interim results of the PBCAR0191 study are presented in FIGS. 2-15, including data from both CD19 CAR T-naïve subjects, and six subjects (5 NHL, 1 B-ALL) who had previously received CD19-directed autologous CAR T therapy. In a cohort of 23 heavily pre-treated NHL and B-ALL subjects, dosing of PBCAR0191 with enhanced lymphodepletion markedly increased PBCAR T cell expansion and AUC resulting in improved ORR and CR rates when compared to subjects receiving standard lymphodepletion. In CAR naïve subjects, the ORR and CR rate was 63% and 56% respectively, comparable to results reported from autologous CAR T programs. Interestingly, the post-autologous CAR T cohort showed a 100% ORR to PBCAR0191 therapy in combination with enhanced lymphodepletion, with 60% and 100% CR rates for NHL and B-ALL subjects, respectively. Furthermore, in 5 evaluable subjects who had relapsed after autologous CAR T therapy, more than 50% of those subjects have had longer duration of response to PBCAR0191 as compared to autologous CAR T therapy. Three of those subjects remain in clinical response post-PBCAR0191 dosing. Collectively, the expansion kinetics and resulting clinical responses seen in subjects who have relapsed after autologous CAR T therapy provides a potential efficacy signal for subjects with high unmet need due to limited therapeutic options.

### Example 3

### Interim Results of PBCAR0191 Study Including Treatment of Patients Who Previously Received Autologous CD19-Directed CAR T Therapy, Utilizing Modified Lymphodepletion Regimen

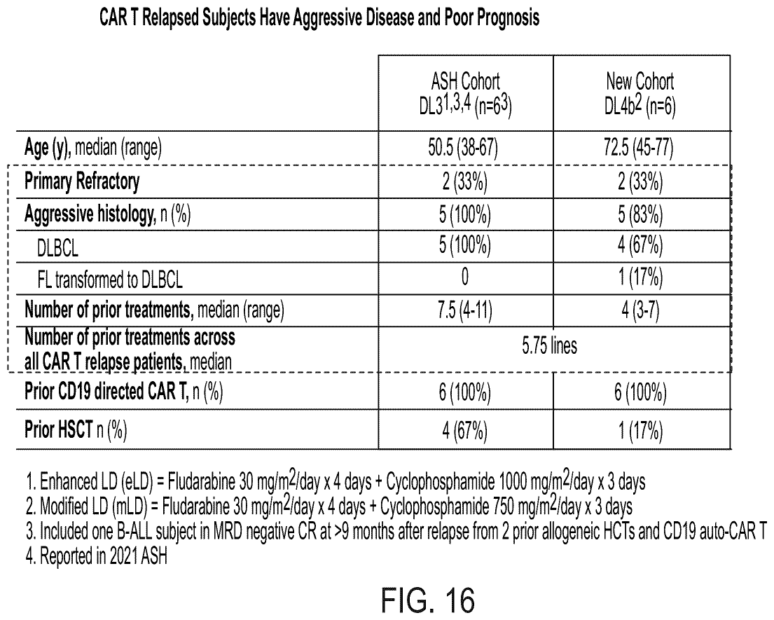

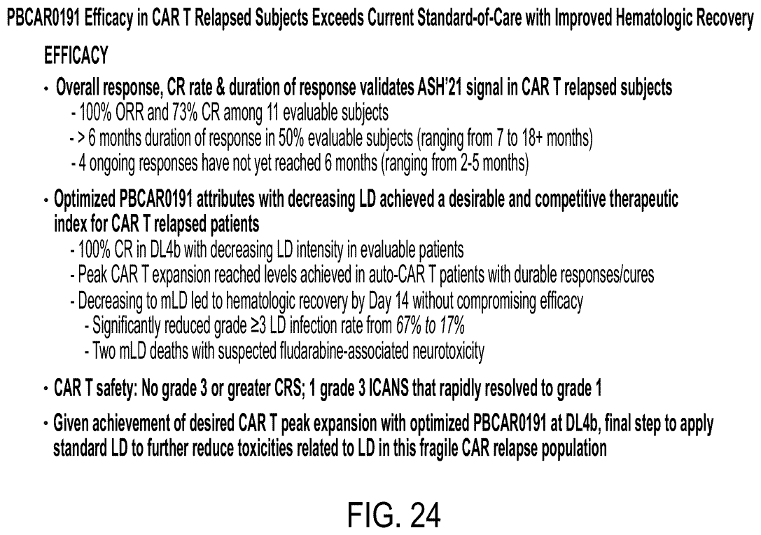

Further interim results of the PBCAR0191 study are presented in FIGS. 16-24 for subjects having diffuse large B-cell lymphoma (DLBCL) or follicular lymphoma transformed to DLBCL. All subjects had previously received CD19-directed autologous CAR T therapy, achieved a response, and then relapsed. The characteristics of CTM material utilized in PBCAR0191 is shown in FIG. 11.

Six subjects discussed in these interim results were subjects treated in Example 2, who received an enhanced lymphodepletion (eLD) regimen that included the administration of cyclophosphamide (1000 mg/m²/d) on days −5 to −3 and fludarabine (30 mg/m²/d) on days −6 to −3 prior to PBCAR0191. Each eLD-treated subject received a DL3 dose of PBCAR0191 anti-CD19 CAR T cells (3.0×10⁶/kg) on day 0. An additional six subjects discussed in these interim results received a modified lymphodepletion (mLD) regimen, which included the administration of cyclophosphamide (750 mg/m²/d) on days −5 to −3 and fludarabine (30 mg/m²/d) on days −6 to −3 prior to PBCAR0191 infusion. Each mLD-treated subject received a DL4b dose of PBCAR0191 anti-CD19 CAR T cells (500×10⁶ flat dose) on day 0. Subject characteristics are shown in FIG. 16.

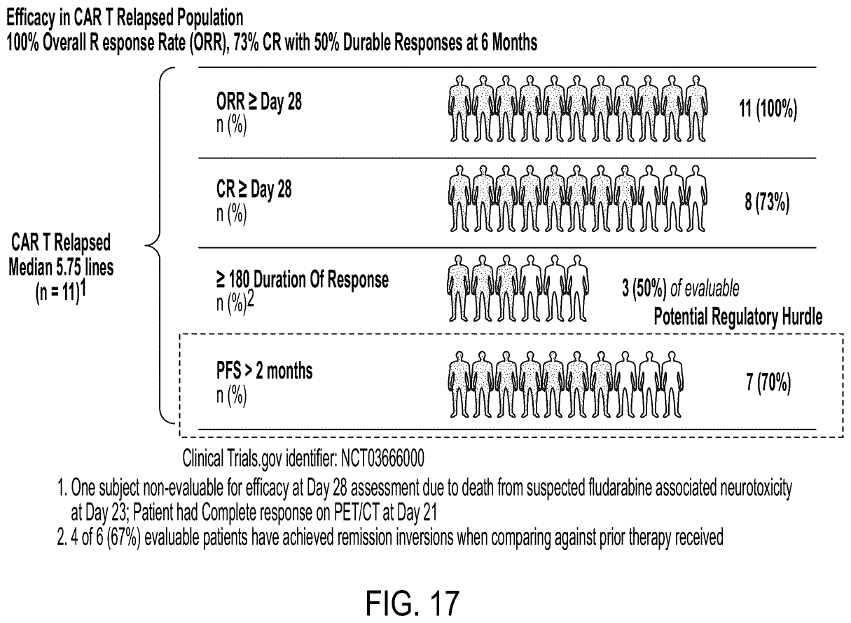

Across both regimens, an overall response rate (ORR) of 100% at day 28 post-CAR T administration was observed in 11 evaluable subjects (11/11), with a complete response rate (CR) observed in 73% of evaluable subjects (8/11). The duration of response was greater than 180 days in 50% of evaluable subjects (3/6) and progression-free survival (PFS) of greater than 2 months was observed in 70% of evaluable subjects (7/10). See, FIG. 17.

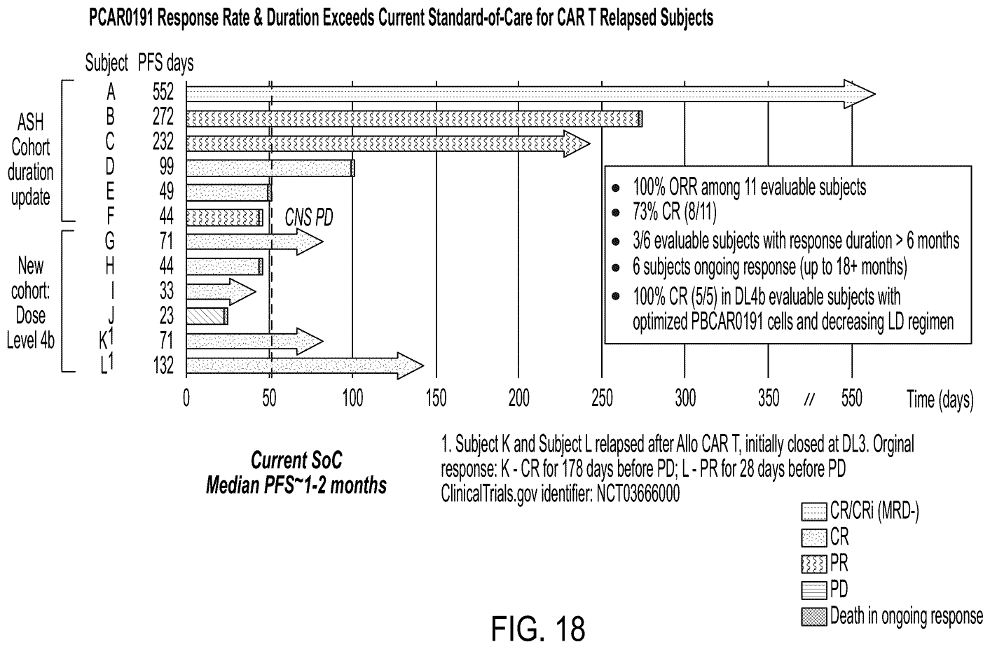

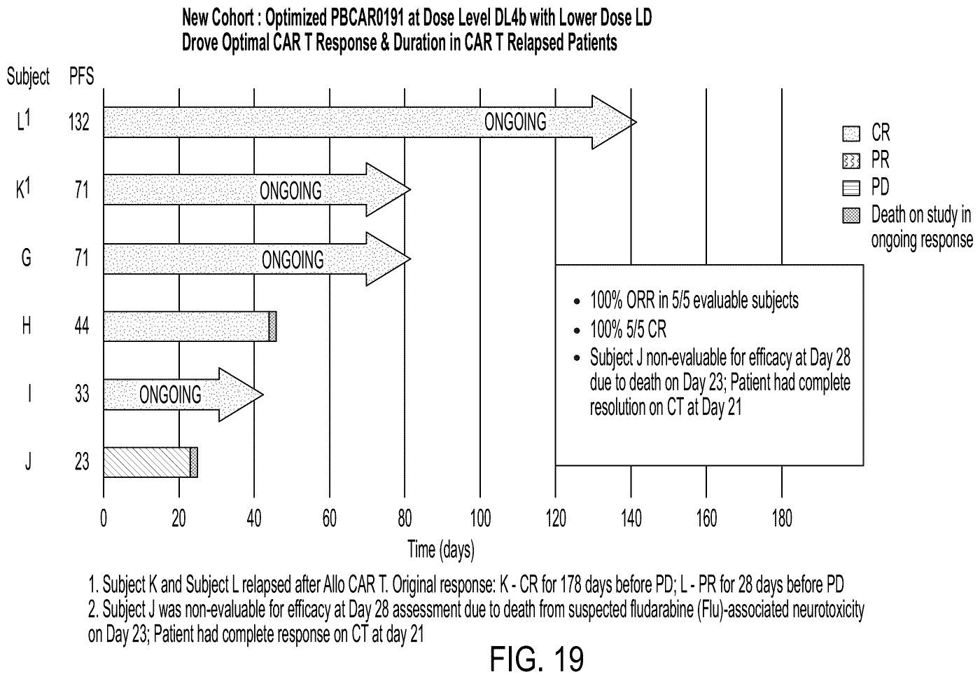

Of the subjects receiving eLD and dose level DL3 (Subjects A-F, FIG. 18), a 100% ORR was observed, with a 50% CR. One subject receiving eLD and dose level DL3 produced a complete remission with incomplete count recovery (CRi) and was MRD-negative. Of the subjects receiving mLD and dose level DL4b (Subjects G-K, FIG. 18 and FIG. 19), a 100% ORR was observed, with a 100% CR in all evaluable subjects. Subjects K and L had previously relapsed after a prior allogeneic CAR T regimen and were retreated with mLD and DL4b, which generated a CR in both subjects.

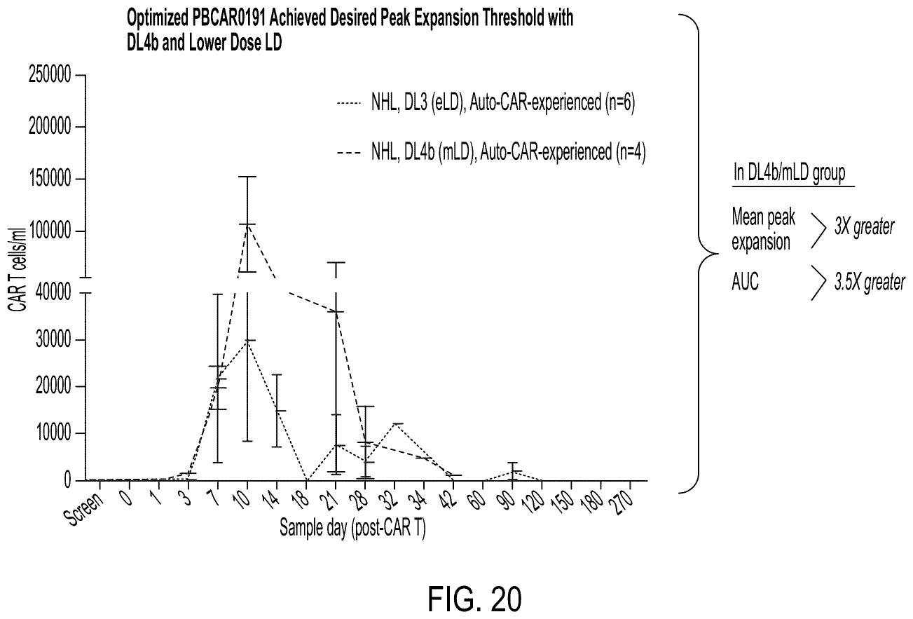

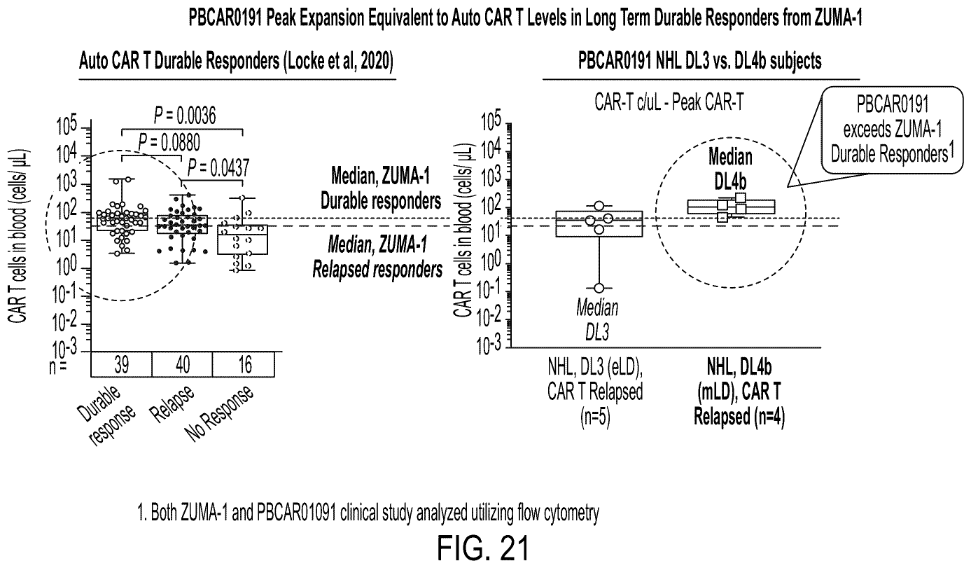

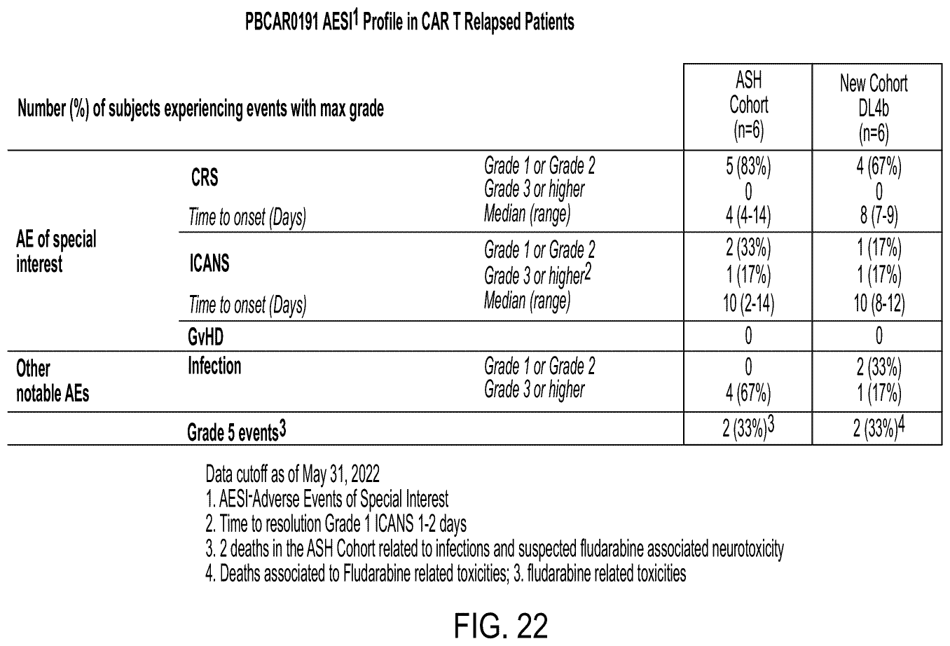

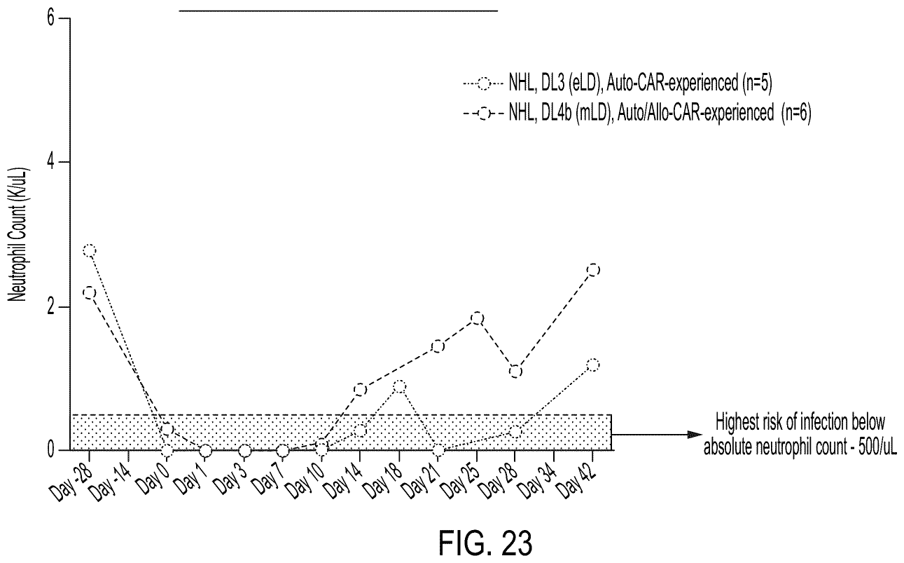

Peak in vivo CAR T cell expansion was analyzed by flow cytometry following administration, and it was observed that the mean peak expansion and area under the curve (AUC) were both elevated in the mLD/DLb4 group compared to the eLD/DL3 group (FIG. 20). Notably, the median peak number of CAR T cells in blood observed in the mLD/DLb4 group exceeded median peak numbers of CAR T cells observed in subjects receiving FDA-approved autologous CAR T therapies that produced durable responses (FIG. 21). Safety profiles observed for each regimen are shown in FIG. 22. Furthermore, FIG. 23 shows that median hematologic recovery was achieved at an earlier time point in mLD/DL4b-treated subjects compared to subjects receiving the most intense eLD regimen.

Collectively, the clinical responses and CAR T cell expansion kinetics observed in these subjects, who have relapsed after autologous CAR T therapy, provides a potential efficacy signal for subjects with high unmet need due to limited therapeutic options.

Sequence Listing

SEQ ID NO: 1
TGGCCTGGAGCAACAAATCTGA
SEQ ID NO: 2
ACCGGACCTCGTTGTTTAGACT

1. A method for reducing the number of cancer cells in a subject, said method comprising:
(a) administering to said subject a lymphodepletion regimen that comprises one or more chemotherapeutic lymphodepletion agents; and
(b) administering to said subject an effective dose of a pharmaceutical composition comprising a population of human immune cells;
wherein a plurality of said human immune cells are genetically-modified human immune cells that express an engineered antigen receptor that has specificity for an antigen on said cancer cells,
wherein said lymphodepletion regimen is administered prior to administration of said pharmaceutical composition,
wherein the number of cancer cells is reduced in said subject,
and wherein said subject was previously administered an autologous cell therapy, achieved a response to said autologous cell therapy, and subsequently relapsed, all prior to (a) and (b).

2. A method for treating a cancer in a subject who has relapsed following an autologous cell therapy, said method comprising:
(a) administering to said subject a lymphodepletion regimen that comprises one or more chemotherapeutic lymphodepletion agents; and
(b) administering to said subject an effective dose of a pharmaceutical composition comprising a population of human immune cells;
wherein a plurality of said human immune cells are genetically-modified human immune cells that express an engineered antigen receptor that has specificity for an antigen on cancer cells,
wherein said lymphodepletion regimen is administered prior to administration of said pharmaceutical composition,
and wherein said subject was previously administered an autologous cell therapy, achieved a response to said autologous cell therapy, and subsequently relapsed, all prior to (a) and (b).

3. The method of claim 1 or claim 2, wherein said human immune cells are not derived from said subject.

4. The method of any one of claims 1-3, wherein said method is a method of immunotherapy for treating cancer in said subject.

5. The method of any one of claims 1-4, wherein said autologous cell therapy was directed against said antigen on said cancer cells.

6. The method of any one of claims 1-5, wherein said subject achieved a partial response to said autologous cell therapy.

7. The method of any one of claims 1-5, wherein said subject achieved a complete response to said autologous cell therapy.

8. The method of any one of claims 1-7, wherein said subject received a stem cell transplant prior to relapse.

9. The method of any one of claims 1-8, wherein said subject received an allogeneic stem cell transplant prior to relapse.

10. The method of any one of claims 1-9, wherein said autologous cell therapy comprises an autologous chimeric antigen receptor (CAR) T cell therapy.

11. The method of any one of claims 1-9, wherein said autologous cell therapy comprises an autologous CAR natural killer (CAR NK) cell therapy.

12. The method of any one of claims 1-9, wherein said autologous cell therapy comprises an autologous T cell therapy comprising administration of T cells expressing an exogenous TCR.

13. The method of any one of claims 1-9, wherein said autologous cell therapy comprises an autologous NK cell therapy comprising administration of NK cells expressing an exogenous TCR.

14. The method of any one of claims 1-13, wherein said method is performed within about 15 days, about 30 days, about 45 days, about 60 days, about 75 days, about 90 days, about 120 days, about 150 days, or about 180 days after relapse following said autologous cell therapy.

15. The method of any one of claims 1-14, wherein said subject achieves a response after said method.

16. The method of claim 15, wherein said response is a partial response.

17. The method of claim 15, wherein said response is a complete response.

18. The method of any one of claims 15-17, wherein said response persists for at least 28 days.

19. The method of any one of claims 15-18, wherein said response persists for about 45 days, about 60 days, about 75 days, about 90 days, about 120 days, about 150 days, about 180 days, or more.

20. The method of any one of claims 15-19, wherein said response to said method persists longer than said response to said autologous cell therapy.

21. The method of any one of claims 1-20, wherein said cancer is a hematological cancer.

22. The method of any one of claims 1-21, wherein said cancer is a cancer of B cell origin or multiple myeloma.

23. The method of claim 22, wherein said cancer of B cell origin is acute lymphoblastic leukemia (ALL), chronic lymphocytic leukemia (CLL), small lymphocytic lymphoma (SLL), or non-Hodgkin lymphoma (NHL).

24. The method of claim 21 or claim 22, wherein said cancer is mantle cell lymphoma (MCL) or diffuse large B cell lymphoma (DLBCL).

25. The method of any one of claims 1-20, wherein said cancer is a solid tumor cancer.

26. The method of any one of claims 1-25, wherein said engineered antigen receptor is a CAR or an exogenous TCR having specificity for said antigen.

27. The method of any one of claims 1-26, wherein said antigen is CD19.

28. The method of any one of claims 1-27, wherein said human immune cells are human T cells or human NK cells.

29. The method of any one of claims 1-28, wherein said genetically-modified human immune cells comprise an inactivated TCR alpha gene, TCR alpha constant region (TRAC) gene, TCR beta gene, or TCR beta constant region (TRBC) gene.

30. The method of any one of claims 1-29, wherein a transgene encoding said engineered antigen receptor is inserted into the genome of said genetically-modified immune cells.

31. The method of claim 30, wherein said transgene is inserted within a TCR alpha gene, a TRAC gene, a TCR beta gene, or a TRBC gene, wherein said transgene disrupts expression of said TCR alpha gene, said TRAC gene, said TCR beta gene, or said TRBC gene.

32. The method of claim 30 or claim 31, wherein said transgene is positioned in said TRAC gene within SEQ ID NO: 1.

33. The method of any one of claims 30-32, wherein said transgene is positioned in said TRAC gene between nucleotides 13 and 14 of SEQ ID NO: 1.

34. The method of any one of claims 1-33, wherein said genetically-modified human immune cells do not have detectable cell surface expression of an endogenous alpha/beta TCR.

35. The method of any one of claims 1-34, wherein said genetically-modified human immune cells do not have detectable cell surface expression of endogenous CD3.

36. The method of any one of claims 1-35, wherein said population of human immune cells comprises one or more of the following characteristics:
(i) said genetically-modified human immune cells represent between about 40% and 75% of cells in said population of human immune cells;
(ii) said genetically-modified human immune cells are T cells, and the ratio of CD4+ genetically-modified human T cells to CD8+ genetically-modified human T cells in said population is between about 0.3 and about 4.2;
(iii) said genetically-modified human immune cells are T cells, and the percentage of CD4+ genetically-modified human T cells in said population that are also CCR7+ is between about 23% to about 60%; and
(iv) said genetically-modified human immune cells are T cells, and the percentage of CD8+ genetically-modified human T cells in said population that are also CCR7+ is between about 14% and about 60%.

37. The method of any one of claims 1-36, wherein said genetically-modified human immune cells represent between about 50% and about 70% of said human immune cells in said population.

38. The method of any one of claims 1-37, wherein said genetically-modified human immune cells represent between about 55% and about 70% of said human immune cells in said population.

39. The method of any one of claims 1-38, wherein said genetically-modified human immune cells represent between about 58% and about 69% of said human immune cells in said population.

40. The method of any one of claims 1-39, wherein no more than about 0.5% of said human immune cells in said population have detectable cell surface expression of CD3.

41. The method of any one of claims 1-40, wherein no more than about 0.3% of said human immune cells in said population have detectable cell surface expression of CD3.

42. The method of any one of claims 1-41, wherein no more than about 0.2% of said human immune cells in said population have detectable cell surface expression of CD3.

43. The method of any one of claims 1-42, wherein no human immune cells in said population have detectable cell surface expression of CD3.

44. The method of any one of claims 36-43, wherein the ratio of CD4+ genetically-modified human T cells to CD8+ genetically-modified human T cells in said population is between about 0.3 and about 4.2.

45. The method of any one of claims 36-44, wherein the percentage of CD4+ genetically-modified human T cells in said population that are also CCR7+ is between about 20% to about 70%.

46. The method of any one of claims 36-45, wherein the percentage of CD4+ genetically-modified human T cells in said population that are also CCR7+ is between about 23% to about 60%.

47. The method of any one of claims 36-46, wherein the percentage of CD8+ genetically-modified human T cells in said population that are also CCR7+ is between about 10% and about 60%.

48. The method of any one of claims 36-47, wherein the percentage of CD8+ genetically-modified human T cells in said population that are also CCR7+ is between about 14% and about 60%.

49. The method of any one of claims 1-48, wherein said one or more chemotherapeutic agents comprises an alkylating agent.

50. The method of claim 49, wherein said alkylating agent is cyclophosphamide.

51. The method of any one of claims 1-50, wherein said one or more chemotherapeutic agents comprises a nucleoside analog.

52. The method of claim 51, wherein said nucleoside analog is fludarabine.

53. The method of any one of claims 49-52, wherein said lymphodepletion regimen comprises administering said alkylating agent at a dose of about 400 to about 1500 mg/m²/day.

54. The method of any one of claims 49-53, wherein said lymphodepletion regimen comprises administering said alkylating agent at a dose of about 500 to about 1000 mg/m²/day.

55. The method of any one of claims 49-54, wherein said lymphodepletion regimen comprises administering said alkylating agent at a dose of about 600 to about 800 mg/m²/day.

56. The method of any one of claims 49-54, wherein said lymphodepletion regimen comprises administering said alkylating agent at a dose of about 500 mg/m²/day.

57. The method of any one of claims 49-54, wherein said lymphodepletion regimen comprises administering said alkylating agent at a dose of about 750 mg/m²/day.

58. The method of any one of claims 49-54, wherein said lymphodepletion regimen comprises administering said alkylating agent at a dose of about 1000 mg/m²/day.

59. The method of any one of claims 51-58, wherein said lymphodepletion regimen comprises administering said nucleoside analog at a dose of about 10 to about 40 mg/m²/day.

60. The method of any one of claims 51-59, wherein said lymphodepletion regimen comprises administering said nucleoside analog at a dose of about 20 to about 40 mg/m²/day.

61. The method of any one of claims 51-60, wherein said lymphodepletion regimen comprises administering said nucleoside analog at a dose of about 30 to about 40 mg/m²/day.

62. The method of any one of claims 51-61, wherein said lymphodepletion regimen comprises administering said nucleoside analog at a dose of about 30 mg/m²/day.

63. The method of claim 51 or claim 52, wherein said lymphodepletion regimen comprises administering said alkylating agent at a dose of about 500 mg/m²/day and said nucleoside analog at a dose of about 30 mg/m²/day.

64. The method of claim 51 or claim 52, wherein said lymphodepletion regimen comprises administering said alkylating agent at a dose of about 750 mg/m²/day and said nucleoside analog at a dose of about 30 mg/m²/day.

65. The method of claim 51 or claim 52, wherein said lymphodepletion regimen comprises administering said alkylating agent at a dose of about 1000 mg/m²/day and said nucleoside analog at a dose of about 30 mg/m²/day.

66. The method of any one of claims 49-65, wherein said lymphodepletion regimen comprises administering said alkylating agent daily starting 5 days and ending 3 days prior to administration of said pharmaceutical composition.

67. The method of any one of claims 51-66, wherein said lymphodepletion regimen comprises administering said nucleoside analog daily starting 5 days and ending 3 days prior to administration of said pharmaceutical composition.

68. The method of any one of claims 51-66, wherein said lymphodepletion regimen comprises administering said nucleoside analog daily starting 6 days and ending 3 days prior to administration of said pharmaceutical composition.

69. The method of any one of claims 51-67, wherein said lymphodepletion regimen comprises administering said alkylating agent and said nucleoside analog daily starting 5 days and ending 3 days prior to administration of said pharmaceutical composition.

70. The method of any one of claims 51-66, wherein said lymphodepletion regimen comprises administering said alkylating agent daily starting 5 days and ending 3 days prior to administration of said pharmaceutical composition, and administering said nucleoside analog daily starting 6 days and ending 3 days prior to administration of said pharmaceutical composition.

71. The method of claim 51 or claim 52, wherein said lymphodepletion regimen comprises administering said alkylating agent at a dose of about 500 mg/m²/day and said nucleoside analog at a dose of about 30 mg/m²/day daily starting 5 days and ending 3 days prior to administration of said pharmaceutical composition.

72. The method of claim 51 or claim 52, wherein said lymphodepletion regimen comprises administering said alkylating agent at a dose of about 750 mg/m²/day and said nucleoside analog at a dose of about 30 mg/m²/day daily starting 5 days and ending 3 days prior to administration of said pharmaceutical composition.

73. The method of claim 51 or claim 52, wherein said lymphodepletion regimen comprises administering said alkylating agent at a dose of about 1000 mg/m²/day and said nucleoside analog at a dose of about 30 mg/m²/day daily starting 5 days and ending 3 days prior to administration of said pharmaceutical composition.

74. The method of claim 51 or claim 52, wherein said lymphodepletion regimen comprises administering said alkylating agent at a dose of about 500 mg/m²/day daily starting 5 days and ending 3 days prior to administration of said pharmaceutical composition, and administering said nucleoside analog at a dose of about 30 mg/m²/day daily starting 6 days and ending 3 days prior to administration of said pharmaceutical composition.

75. The method of claim 51 or claim 52, wherein said lymphodepletion regimen comprises administering said alkylating agent at a dose of about 750 mg/m²/day daily starting 5 days and ending 3 days prior to administration of said pharmaceutical composition, and administering said nucleoside analog at a dose of about 30 mg/m²/day daily starting 6 days and ending 3 days prior to administration of said pharmaceutical composition.

76. The method of claim 51 or claim 52, wherein said lymphodepletion regimen comprises administering said alkylating agent at a dose of about 1000 mg/m²/day daily starting 5 days and ending 3 days prior to administration of said pharmaceutical composition, and administering said nucleoside analog at a dose of about 30 mg/m²/day daily starting 6 days and ending 3 days prior to administration of said pharmaceutical composition.

77. The method of any one of claims 1-76, wherein said pharmaceutical composition is administered at a dose of between about 1×10⁵ and about 6×10⁶ genetically-modified human immune cells/kg.

78. The method of any one of claims 1-77, wherein said pharmaceutical composition is administered at a dose of between about 3×10⁵ and about 6×10⁶ genetically-modified human immune cells/kg.

79. The method of any one of claims 1-78, wherein said pharmaceutical composition is administered at a dose of between about 3×10⁵ and about 3×10⁶ genetically-modified human immune cells/kg.

80. The method of any one of claims 1-79, wherein said pharmaceutical composition is administered at a dose of between about 1×10⁶ and about 3×10⁶ genetically-modified human immune cells/kg.

81. The method of any one of claims 1-80, wherein said pharmaceutical composition is administered at a dose of about 1×10⁶, about 1.5×10⁶, about 2×10⁶, about 2.5×10⁶, or about 3×10⁶ genetically-modified human immune cells/kg, or at a dose of about 200×10⁶, about 300×10⁶, about 400×10⁶, about 500×10⁶, about 600×10⁶, about 700×10⁶, about 800×10⁶, about 900×10⁶, or about 1×10⁷ genetically-modified human immune cells.

82. The method of any one of claims 1-81, wherein said lymphodepletion regimen includes no greater than a minimal effective dose of a biological lymphodepletion agent.

83. The method of any one of claims 1-82, wherein said lymphodepletion regimen includes administration of a biological lymphodepletion agent in an amount no greater than 1.0 mg/kg during the 7 day period preceding administration of said pharmaceutical composition.

84. The method of any one of claims 1-83, wherein said lymphodepletion regimen does not include administration of a biological lymphodepletion agent.

85. The method of any one of claims 1-81, wherein said lymphodepletion regimen includes administration of a biological lymphodepletion agent.

86. The method of any one of claims 82-85, wherein said biological lymphodepletion agent is a monoclonal antibody, or a fragment thereof.

87. The method of claim 86, wherein said monoclonal antibody, or fragment thereof, has specificity for a T cell antigen.

88. The method of claim 87, wherein said monoclonal antibody, or fragment thereof, is an anti-CD52 monoclonal antibody, or fragment thereof, or an anti-CD3 antibody, or fragment thereof.

89. The method of any one of claims 1-88, wherein said method reduces the size of a cancer or tumor in said subject.

90. The method of any one of claims 1-89, wherein said method eradicates said cancer or tumor in said subject.
